(19) United States
(12) Patent Application Publication
(10) Pub. No.: US 2024/0130871 A1
(43) Pub. Date: Apr. 25, 2024

(54) DRUG-COATED ENDOVASCULAR DEVICES

(71) Applicant: UNIVERSITY OF SOUTH CAROLINA, COLUMBIA, SC (US)

(72) Inventors: TAREK SHAZLY, COLUMBIA, SC (US); VIJAYA B. KOLACHALAMA, BOSTON, MA (US)

(21) Appl. No.: 18/488,359

(22) Filed: Oct. 17, 2023

Related U.S. Application Data
(60) Provisional application No. 63/416,718, filed on Oct. 17, 2022.

(51) Int. Cl. A61F 2/82; A61M 25/10

(52) U.S. Cl. CPC A61F 2/82; A61M 25/10; A61F 2250/0067; A61M 2025/105

(57) ABSTRACT

In general, the present disclosure is directed to an endovascular device. The device may include an outer body comprising a biocompatible polymeric material; and a core comprising a first layer and a second layer, wherein the first layer comprising an anti-contractile agent and the second layer comprising a second agent.

### CROSS REFERENCE TO RELATED APPLICATIONS

This application claims filing benefit of U.S. Provisional Patent Application Ser. No. 63/416,718, filed on Oct. 17, 2022, which is incorporated herein by reference.

### GOVERNMENT SUPPORT CLAUSE

This invention was made with government support under grant R01 HL159620 awarded by National Institutes of Health (NIH). The government has certain rights in the invention.

### BACKGROUND

Drug-eluting stents (DES), while certainly a successful endovascular device class, are nevertheless limited by delayed healing, protracted/incomplete reendothelialization, in-stent thrombosis, and stent fracture. Several of these issues are inherently addressed by the leave-nothing-behind strategy embodied in drug coated balloon (DCB) therapy, in which a transient (˜2-3 minutes) intravascular balloon inflation both compresses the lesion site and locally delivers an anti-proliferative drug, most commonly paclitaxel (PTX), to the arterial wall. Thus, with no permanent implant, DCBs offer a means to acutely restore (via mechanical compression) and subsequently maintain (via PTX activity) vessel patency. DCBs have seen extensive use in the treatment of peripheral artery disease (PAD), which affects over 8 million Americans and carries an increased risk for African Americans, and as such represent a device class of high clinical significance. However, the manifest regulation of these devices in the face of controversial analyses that associates endovascular PTX delivery with increased mortality risk, their comparatively sparse use in the coronary circulation, and an incomplete understanding of the key determinants of therapeutic efficacy, together indicate that DCB clinical potential has not been fully realized.

Clinical hesitation for broader DCB use can be attributed to inefficient component transfer from the device to the arterial wall and inconsistent/unpredictable patient outcomes. Indeed, current devices transfer only a small fraction of coating (<10%) and most (>90%) of the delivered PTX is lost to systemic circulation—this leads to ineffective and unpredictable therapy and increases the risk of systemic toxicity. These shortcomings may be addressed in at least two fundamental ways: 1) increasing the efficiency of DCB intra-procedural drug transfer via strategic coating/balloon design and 2) manipulating the post-DCB local biological response via an alternate drug payload.

Based on experimental observations of maladaptive arterial remodeling outcomes, including positive associations among vascular smooth muscle cells (vSMC) contractility, endothelial cell (EC) dysfunction, and inward vessel remodeling, post-procedural induction of vSMC relaxation may shift the direction of remodeling-mediated growth from inward to outward and insomuch prevent subsequent loss of lumen patency. This notion is further supported by our preliminary theoretical studies that predict the role vSMC phenotype in arterial remodeling in the presence of EC dysfunction, where the ratio between the synthetic and contractile vSMCs is a deterministic factor for remodeling outcomes, including the wall growth direction. EC dysfunction, which may be pre-existent and/or exacerbated during lesion preparation, could vary on a patient-specific basis, and thus partially underlie inconsistent outcomes in DCB therapy.

As such, there is a need in the art for materials and methods for treating peripheral artery disease. Particularly, materials and methods capable of treating peripheral artery disease in a subject in need thereof and preserving the lumen upon remodeling completion.

### SUMMARY

In general, the present disclosure is directed to an endovascular device. The device may include an outer body comprising a biocompatible polymeric material; and a core comprising a first layer and a second layer, wherein the first layer comprising an anti-contractile agent and the second layer comprising a second agent.

Additionally, the present disclosure is directed to methods for treating peripheral artery disease in a subject in need thereof. The method may include implanting an endovascular device within a peripheral vessel of the subject, wherein the endovascular device comprises an outer body comprising a biocompatible polymeric material and a core comprising a first layer and a second layer, wherein the first layer comprising an anti-contractile agent and the second layer comprising a second agent; releasing from the endovascular device a therapeutically effective amount of the anti-contractile agent and the second drug, wherein release of the anti-contractile agent in combination with the second drug preserves the lumen upon maladaptive inward remodeling completion in the subject.

Also, the present disclosure is directed to a modeling framework for predicting the maladaptive remodeling effects of delivery of an anti-proliferative agent to a blood vessel by use of a drug coated balloon (DCB), the modeling framework utilizing the following governing equations:

$$\frac{{P}^{H} {r}_{i ( m a )}}{{h}_{( m a )}} - {σ}_{2 ( b )} = 0$$

$${S}_{( m a )} = \{ \begin{matrix}{S}_{( b )} + ( {S}_{( max )} - {S}_{( b )} ) \frac{{P}^{H} - {P}^{N}}{{P}^{c r} - {P}^{N}} & if {P}^{H} \leq {P}^{cr} \\ {S}_{( max )} & if {P}^{H} > {P}^{cr}\end{matrix}$$

$$2 π ( {r}_{i ( m a )} + \frac{{h}_{( m a )}}{2} ) {h}_{( m a )} - {a}_{( b )} + ( \frac{{S}_{( max )} - {S}_{( m a )}}{{S}_{( max )} - {S}_{( b )}} ) ( 1 - {D}_{[ A P ]} ) ( {a}_{( b )} - {a}_{( a )} ) = 0$$

in which:
p^{H} refers to the hypertensive pressure within the vessel,
p^{N} refers to the normotensive pressure within the vessel,
p^{cr} prefers to a critical pressure,
rᵢ refers to the inner radius of the vessel,
s₂ refers to the value for equilibrium in the radial direction of the vessel,
h refers to the vessel wall thickness,
ma (subscript) refers to maladaptive post-DCB remodeling outcomes,
Sₘₐₓ refers to the maximal contractile capacity of the smooth muscle cells of the vessel at a pressure equal to or greater than p^{cr},
S_{b} refers to the basal contractile value of the smooth muscle cells of the vessel at p^{N},
Sₘₐ refers to the maladaptive post-DCB remodeling contractile value of the smooth muscle cells of the vessel,
aₐ refers to the cross-sectional wall area of the remodeled artery adaptive remodeling outcome,
a_{b} refers to the basal cross-sectional wall area, and
D_{[AP]} refers to the normalized dosing parameter of the anti-proliferative agent.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1A illustrates pressure-outer diameter data of the porcine femoral artery when the artery is at fixed axial stretch (λ₁=2.0) and under basal, maximally contracted, and fully relaxed SMC states.

FIG. 1B illustrates axial force-pressure relations of the porcine femoral artery when the artery is at fixed axial stretch (λ₁=2.0) and under basal, maximally contracted, and fully relaxed SMC states.

FIG. 1C illustrates histological preparations to assess arterial wall composition include (i) Resorcin Fuchsin with Woodstain Scarlet Acid Fuchsin counterstain, Direct Red (0.1% in saturated picric acid) using (ii) bright-field, and (iii) cross-polarized light, and (iv) a combination of Verhoeff-Masson's stain.

FIG. 2A illustrates the total, active, and passive circumferential stress-circumferential stretch relations when the artery is at fixed axial stretch (λ₁=2.0) and under basal and (b) fully contracted SMC states. Points indicate experimental data; curves indicate fits with identified constitutive models.

FIG. 2B illustrates the total, active, and passive circumferential stress-circumferential stretch relations when the artery is at fixed axial stretch (λ₁=2.0) and under fully contracted SMC states. Points indicate experimental data; curves indicate fits with identified constitutive models.

FIG. 3A illustrates atrial geometry data of undeformed inner radius of adaptive pressure-mediated arterial remodeling following traditional DCB deployment. Remodeling outcomes for AP dosing parameter (D_{[AP]}) ranging from 0-1 in 0.2 increments, with the arrow indicating the dosing associated with each curve.

FIG. 3B illustrates atrial geometry data of undeformed wall thickness of adaptive pressure-mediated arterial remodeling following traditional DCB deployment. Remodeling outcomes for AP dosing parameter (D_{[AP]}) ranging from 0-1 in 0.2 increments, with the arrow indicating the dosing associated with each curve.

FIG. 3C illustrates atrial geometry data of undeformed wall area of adaptive pressure-mediated arterial remodeling following traditional DCB deployment. Remodeling outcomes for AP dosing parameter (D_{[AP]}) ranging from 0-1 in 0.2 increments, with the arrow indicating the dosing associated with each curve.

FIG. 3D illustrates atrial geometry data of deformed inner radius of adaptive pressure-mediated arterial remodeling following traditional DCB deployment. Remodeling outcomes for AP dosing parameter (D_{[AP]}) ranging from 0-1 in 0.2 increments, with the arrow indicating the dosing associated with each curve.

FIG. 3E illustrates atrial geometry data of deformed wall thickness of adaptive pressure-mediated arterial remodeling following traditional DCB deployment. Remodeling outcomes for AP dosing parameter (D_{[AP]}) ranging from 0-1 in 0.2 increments, with the arrow indicating the dosing associated with each curve.

FIG. 3F illustrates atrial geometry data of deformed wall area of adaptive pressure-mediated arterial remodeling following traditional DCB deployment. Remodeling outcomes for AP dosing parameter (D_{[AP]}) ranging from 0-1 in 0.2 increments, with the arrow indicating the dosing associated with each curve.

FIG. 3G illustrates atrial elastin mass fraction data of adaptive pressure-mediated arterial remodeling following traditional DCB deployment. Remodeling outcomes for AP dosing parameter (D_{[AP]}) ranging from 0-1 in 0.2 increments, with the arrow indicating the dosing associated with each curve.

FIG. 3H illustrates atrial collagen mass fraction data of adaptive pressure-mediated arterial remodeling following traditional DCB deployment. Remodeling outcomes for AP dosing parameter (D_{[AP]}) ranging from 0-1 in 0.2 increments, with the arrow indicating the dosing associated with each curve.

FIG. 3I illustrates atrial SMC mass fraction data of adaptive pressure-mediated arterial remodeling following traditional DCB deployment. Remodeling outcomes for AP dosing parameter (D_{[AP]}) ranging from 0-1 in 0.2 increments, with the arrow indicating the dosing associated with each curve.

FIG. 4A illustrates atrial geometry data of undeformed inner radius of maladaptive pressure-mediated arterial remodeling following traditional DCB deployment. Remodeling outcomes for AP dosing parameter (D_{[AP]}) ranging from 0-1 in 0.2 increments, with the arrow indicating the dosing associated with each curve.

FIG. 4B illustrates atrial geometry data of undeformed wall thickness of maladaptive pressure-mediated arterial remodeling following traditional DCB deployment. Remodeling outcomes for AP dosing parameter (D_{[AP]}) ranging from 0-1 in 0.2 increments, with the arrow indicating the dosing associated with each curve.

FIG. 4C illustrates atrial geometry data of undeformed wall area of maladaptive pressure-mediated arterial remodeling following traditional DCB deployment. Remodeling outcomes for AP dosing parameter (D_{[AP]}) ranging from 0-1 in 0.2 increments, with the arrow indicating the dosing associated with each curve.

FIG. 4D illustrates atrial geometry data of deformed inner radius of maladaptive pressure-mediated arterial remodeling following traditional DCB deployment. Remodeling outcomes for AP dosing parameter (D_{[AP]}) ranging from 0-1 in 0.2 increments, with the arrow indicating the dosing associated with each curve.

FIG. 4E illustrates atrial geometry data of deformed wall thickness of maladaptive pressure-mediated arterial remodeling following traditional DCB deployment. Remodeling outcomes for AP dosing parameter (D_{[AP]}) ranging from 0-1 in 0.2 increments, with the arrow indicating the dosing associated with each curve.

FIG. 4F illustrates atrial geometry data of deformed wall area of maladaptive pressure-mediated arterial remodeling following traditional DCB deployment. Remodeling outcomes for AP dosing parameter (D_{[AP]}) ranging from 0-1 in 0.2 increments, with the arrow indicating the dosing associated with each curve.

FIG. 4G illustrates atrial elastin mass fraction data of maladaptive pressure-mediated arterial remodeling following traditional DCB deployment. Remodeling outcomes for AP dosing parameter (D_{[AP]}) ranging from 0-1 in 0.2 increments, with the arrow indicating the dosing associated with each curve.

FIG. 4H illustrates atrial collagen mass fraction data of maladaptive pressure-mediated arterial remodeling following traditional DCB deployment. Remodeling outcomes for AP dosing parameter (D_{[AP]}) ranging from 0-1 in 0.2 increments, with the arrow indicating the dosing associated with each curve.

FIG. 4I illustrates atrial SMC mass fraction data of maladaptive pressure-mediated arterial remodeling following traditional DCB deployment. Remodeling outcomes for AP dosing parameter (D_{[AP]}) ranging from 0-1 in 0.2 increments, with the arrow indicating the dosing associated with each curve.

FIG. 5A illustrates atrial geometry data of undeformed inner radius of maladaptive pressure-mediated arterial remodeling following traditional DCB deployment. Remodeling outcomes for a fixed AP dosing parameter (D_{[AP]}=0.5) with the AC dosing parameter (D_{[AP]}) ranging from 0-1 in 0.2 increments, with the arrow indicating the dosing associated with each curve.

FIG. 5B illustrates atrial geometry data of undeformed wall thickness of maladaptive pressure-mediated arterial remodeling following traditional DCB deployment. Remodeling outcomes for a fixed AP dosing parameter (D_{[AP]}=0.5) with the AC dosing parameter (D_{[AP]}) ranging from 0-1 in 0.2 increments, with the arrow indicating the dosing associated with each curve.

FIG. 5C illustrates atrial geometry data of undeformed wall area of maladaptive pressure-mediated arterial remodeling following traditional DCB deployment. Remodeling outcomes for a fixed AP dosing parameter (D_{[AP]}=0.5) with the AC dosing parameter (D_{[AP]}) ranging from 0-1 in 0.2 increments, with the arrow indicating the dosing associated with each curve.

FIG. 5D illustrates atrial geometry data of deformed inner radius of maladaptive pressure-mediated arterial remodeling following traditional DCB deployment. Remodeling outcomes for a fixed AP dosing parameter (D_{[AP]}=0.5) with the AC dosing parameter (D_{[AP]}) ranging from 0-1 in 0.2 increments, with the arrow indicating the dosing associated with each curve.

FIG. 5E illustrates atrial geometry data of deformed wall thickness of maladaptive pressure-mediated arterial remodeling following traditional DCB deployment. Remodeling outcomes for a fixed AP dosing parameter (D_{[AP]}=0.5) with the AC dosing parameter (D_{[AP]}) ranging from 0-1 in 0.2 increments, with the arrow indicating the dosing associated with each curve.

FIG. 5F illustrates atrial geometry data of deformed wall area of maladaptive pressure-mediated arterial remodeling following traditional DCB deployment. Remodeling outcomes for a fixed AP dosing parameter (D_{[AP]}=0.5) with the AC dosing parameter (D_{[AP]}) ranging from 0-1 in 0.2 increments, with the arrow indicating the dosing associated with each curve.

FIG. 5G illustrates atrial elastin mass fraction data of maladaptive pressure-mediated arterial remodeling following traditional DCB deployment. Remodeling outcomes for a fixed AP dosing parameter (D_{[AP]}=0.5) with the AC dosing parameter (D_{[AP]}) ranging from 0-1 in 0.2 increments, with the arrow indicating the dosing associated with each curve.

FIG. 5H illustrates atrial collagen mass fraction data of maladaptive pressure-mediated arterial remodeling following traditional DCB deployment. Remodeling outcomes for a fixed AP dosing parameter (D_{[AP]}=0.5) with the AC dosing parameter (D_{[AP]}) ranging from 0-1 in 0.2 increments, with the arrow indicating the dosing associated with each curve.

FIG. 5I illustrates atrial SMC mass fraction data of maladaptive pressure-mediated arterial remodeling following traditional DCB deployment. Remodeling outcomes for a fixed AP dosing parameter (D_{[AP]}=0.5) with the AC dosing parameter (D_{[AP]}) ranging from 0-1 in 0.2 increments, with the arrow indicating the dosing associated with each curve.

FIG. 6A illustrates predictions for the deformed wall area of the femoral artery after completion of pressure-mediated remodeling, where Case 1 refers to adaptive remodeling, Case 2 refers to maladaptive remodeling, Case 3 refers to maladaptive post-DCB remodeling with D_{[AP]}=0.5 (traditional DCB with intermediate drug dose), and Case 4 refers to maladaptive post-DCB remodeling with D_{[AP]}=0.5 and D_{[AP]}=0.5 (novel DCB with intermediate drug dose).

FIG. 6B illustrates predictions for the deformed inner radius of the femoral artery after completion of pressure-mediated remodeling, where Case 1 refers to adaptive remodeling, Case 2 refers to maladaptive remodeling, Case 3 refers to maladaptive post-DCB remodeling with D_{[AP]}=0.5 (traditional DCB with intermediate drug dose), and Case 4 refers to maladaptive post-DCB remodeling with D_{[AP]}=0.5 and D_{[AP]}=0.5 (novel DCB with intermediate drug dose).

FIG. 6C illustrates predictions for the vessel pressure-outer diameter relations after completion of remodeling under an intermediate degree of hypertension (P^{H}=150 mmHg) for Cases 1-4; dashed line indicates the baseline relation.

FIG. 6D illustrates predictions for deformed inner radius of the femoral artery after completion of pressure-mediated remodeling, where Case* refers to maladaptive post-DCB remodeling with D_{[AP]}=0.5 and D_{[AP]}=0.74 (novel DCB with tuned drug dose).

FIG. 7A illustrates a spray-based deposition of urea+PTX coatings resulting in fiber-like surface morphology with an aggregate domain.

FIG. 7B illustrates a spray-based deposition of urea+PTX coatings resulting in fiber-like surface morphology with aggregate and diffuse domains.

FIG. 7C illustrates ultraviolet light/oxidation balloon surface treatment prior to spray coating alters the surface morphological profile.

FIG. 7D illustrates ultraviolet light/oxidation balloon surface treatment prior to spray coating promotes relatively sparse yet large aggregate domain formation.

FIG. 7E illustrates a uniaxial mechanical testing set-up used to create a high-throughput assay of acute PTX transfer to the arterial wall, in which flat arterial specimens are compressed with DCB surfaces under controlled force/duration values that simulate balloon deployment.

FIG. 7F illustrates acute PTX transfer to the arterial wall was assessed with HPLC and significantly increased with surface treatment time. *p<0.05 vs untreated surface.

FIG. 8A illustrates controlled alteration of excipient solid content during coating procedure for urea-based DCBs. Design parameters elicit control of coating microstructure (as visualized via SEM).

FIG. 8B illustrates controlled alteration of excipient solid content during coating procedure for urea-based DCBs. Design parameters elicit control of PTX transfer to arterial tissue (assessed in an ex vivo model of DCB deployment). *indicates p<0.05 vs. PTX alone (0% urea), # indicates p<0.05 vs 5% variant.

FIG. 8C illustrates controlled alteration of spray distance during coating procedure for urea-based DCBs. Design parameters elicit control of coating microstructure (as visualized via SEM).

FIG. 8D illustrates controlled alteration of spray distance during coating procedure for urea-based DCBs. Design parameters elicit control of PTX transfer to arterial tissue (assessed in an ex vivo model of DCB deployment). *indicates p<0.05 vs. 7.5 cm variant.

FIG. 9 illustrates subset of structural and mechanical data before (Day-0) and after (Day-21) venous or arterial-like perfusion.

Repeat use of reference characters in the present specification and drawings is intended to represent the same or analogous features or elements of the present invention.

### DETAILED DESCRIPTION

Reference will now be made in detail to various embodiments of the presently disclosed subject matter, one or more examples of which are set forth below. Each embodiment is provided by way of explanation, not limitation, of the subject matter. In fact, it will be apparent to those skilled in the art that various modifications and variations may be made to the present disclosure without departing from the scope or spirit of the disclosure. For instance, features illustrated or described as part of one embodiment, may be used in another embodiment to yield a still further embodiment. Thus, it is intended that the present disclosure cover such modifications and variations as come within the scope of the appended claims and their equivalents.

In general, the present disclosure is directed to an endovascular device for delivering active agents for treating peripheral artery disease. The endovascular device may include an outer body comprising a biocompatible polymeric material; and a core comprising a first layer and a second layer, wherein the first layer comprises an anti-contractile agent and the second layer comprises a second agent.

In one embodiment, the drug coated medical device may be any suitable endovascular device that includes a first layer and a second layer. The drug coated endovascular device may be selected, without limitation, from a group consisting of a balloon catheter, a drainage catheter, a replacement or artificial venous valve, an aortic valve, a replacement valve, a ventricular catheter, a ventriculostomy balloon, a balloon or self-expandable stent, or a coronary balloon.

The endovascular device disclosed herein may be formed using suitable methods known in the art. The endovascular device may be formed of a biocompatible polymeric material. For instance, the endovascular device may be formed from polystyrene, poly(lactic acid), polyketal, butadiene styrene, styrene-acrylic-vinyl terpolymer, poly(methyl methacrylate), poly(ethyl methacrylate), poly(alkyl cyanoacrylate), styrene-maleic anhydride copolymer, poly(vinyl acetate), poly(vinyl pyridine), poly(divinylbenzene), poly(butylene terephthalate), acrylonitrile, vinyl chloride-acrylates, poly(ethylene glycol), and the like, or an aldehyde, carboxyl, amino, hydroxyl, or hydrazide derivative thereof can be utilized. In one embodiment, the endovascular device is made of nylon 12.

Other inorganic biocompatible materials that may be utilized in forming an endovascular device can include, without limitation, oxides such as silica, titania, zirconia, and the like, and noble metals such as gold, silver, platinum, palladium, and the like. In general, the materials will be biocompatible and nonimmunogenic.

The endovascular device disclosed herein may comprise a polymeric material that is biodegradable, biostable, or a combination thereof. For instance, the biodegradable polymeric material may include, but is not limited to, polylactic acid, polyglycolic acid, and their copolymers, caproic acid, polyethylene glycol, polyanhydrides, polyacetates, polycaprolactones, poly(orthoesters), polyamides, polyurethanes, or other suitable polymers. The biostable polymeric material, for instance, may include, but is not limited to, polyethylene, polypropylene, polymethyl methacrylate, polyesters, polyamides, polyurethanes, polytetrafluoroethylene (PTFE), polyvinyl alcohol, or other suitable polymers.

Many factors can be considered in designing and forming the biocompatible material that may be used to influence release of a therapeutic agent from the endovascular device. Exemplary factors when considering a polymer-based scaffold can include, without limitation, molecular weight of polymers, chain length of copolymer components, and ratio of copolymer components. Porosity of the layers can also be formed and designed so as to control degradation rate of a scaffold and/or release rate of a therapeutic agent from a layer.

Selection of the polymeric material can be utilized to provide a primary control of release rate of a therapeutic agent from a loaded layer. For instance, selection of a biodegradable material can be utilized to increase the rate of release and provide a release mechanism that can be limited to a large extent by matrix degradation rate and to a lesser extent by diffusion of the active compound from the bulk material. Alternatively, materials can be utilized such that active compound release rate is limited by only one of diffusion (e.g., a non-degradable matrix) or matrix degradation rate (e.g., essentially no diffusion of the therapeutic agent through the matrix due to small matrix mesh size).

In some embodiments, a drug-coated layer (e.g., one or both of the first and second layer) may include one or more polymorphs of an agent. It is understood that polymorphs may have different solubilities or crystal forms. Polymorphs may have characteristics that affect tissue uptake of the drug at the delivery site or dissolution rate in bodily fluids. The polymorph(s) may be selected to facilitate a particular therapeutic objective.

The first and second layer of the endovascular device may include particles. When incorporated, such particles may have any suitable size profile. For instance, the particular size profile can facilitate uptake of a delivered agent by tissue. In one embodiment, particles can be loaded with a therapeutic agent. In one embodiment, particles can be nanosized and the average diameter of particles can be about 1000 nanometers or less, about 200 nm or less, or about 100 nm or less in some embodiments. Alternatively, a device can utilize larger particulates, such as microparticles having a size of from about 1000 nanometers to about 50 micrometers (μm). Generally, particles are substantially spherical in shape, although other shapes including, but not limited to, plates, rods, bars, irregular shapes, etc., are suitable for use.

The preferred size and shape of the device can depend upon the specific application, e.g., the specific location and size of the tissue in which the device will be implanted as well as the desired release rate of a therapeutic active agent from the layers. For instance, in one embodiment, individual particles containing a therapeutic agent can be adhered to one another to form a larger, optionally porous bulk scaffold for implant. In another embodiment, particles can be of a size and shape so as to be injectable. As will be appreciated by those skilled in the art, the composition, shape, size, and/or density of the endovascular devices may vary widely.

Each drug coated layer of an endovascular device may include an agent in crystalline or amorphous form. For instance, one or both of the first layer and second layer may include a drug in an amorphous form. In another embodiment, one or both of the first layer and second layer may include a drug in a crystalline form. In yet another embodiment, one or both of the first layer and second layer may include a drug in both a crystalline form and an amorphous form.

In one embodiment, the first layer may be coated with an anti-contractile agent. As described herein, an “anti-contractile agent” refers to a drug or drugs administered to prevent or reduce the constriction or contraction of blood vessels. Anti-contractile agents may prevent or reduce blood vessel constriction by relaxing the smooth muscle in the blood vessel walls, thereby keeping the vessels open for adequate blood flow.

The anti-contractile agent may be a nitric oxide donor molecule including, but not limited to, nitroglycerin, isosorbide-5-mononitrate, nicorandil, pentaerythritol tetranitrate, sodium nitroprusside (SNP), S-nitroso-N-acetylpenicillamine (SNAP), S-nitrosoglutathione, molsidomine, spermine NONOate, Proli-NONOate, or a combination thereof.

The anti-contractile agent, for instance, may be an angiotensin receptor blocker (ARB). For instance, the ARB may include, but is not limited to, eprosartan, olmesartan, telmisartan, losartan, valsartan, irbesartan, candesartan, or a combination thereof. In one embodiment, the first layer may be coated with valsartan.

In one embodiment, the anti-contractile agent include, but is not limited to, diltiazem, nimodipine, verapamil, nifedipine, amlodipine, felodipine, isradipine, clevidipine, bepridil, nisoldipine, doxazosin, prazosin, terazosin, alfuzosin, tamsulosin, silodosin, epinephrine, norepinephrine, dopamine, dobutamine, isoproterenol, ouabain, digitalis, digoxin, digitoxin, milrinone, inamrinone, cilostazol, sildenafil, tadalafil, arginine vasopressin terlipressin, warfarin, anisindione, fondaparinux, dalteparin, tinzaparin, enoxaparin, ardeparin, danaparoid, aspirin, prasugrel, cilostazol, clopidogrel, dipyridamole, ticlopidine, argatroban, bivalirudin, desirudin, lepirudin, prednisone, prednisolone, hydrocortisone, nepafenac, ketorolac, flurbiprofen, suprofen, cyclosporine, triamcinolone, diclofenac, bromfena, or a combination thereof.

In one embodiment, the second layer may be coated with an anti-proliferative agent, an anti-inflammatory agent, or a combination thereof. For instance, the second layer may be coated with an anti-proliferative agent or an anti-inflammatory agent. Alternatively, the second layer may be coated with an anti-proliferative agent and an anti-inflammatory agent.

The anti-proliferative agent may be selected from a group consisting of a cytotoxin or a synthetic molecule or other substances such as actinomycin D, or derivatives and analogs thereof; a taxoid such as taxol, docetaxel, and paclitaxel, paclitaxel derivatives; an olimus drug such as macrolide antibiotics, rapamycin, everolimus, novolimus, myolimus, deforolimus, umirolimus, biolimus, merilimus, temsirolimus structural derivatives and functional analogues of rapamycin (such as 40-O-(3-hydroxy)propyl-rapamycin, 40-O-P-(2-hydroxy)ethoxylethyl-rapamycin, 40-O-tetrazole-rapamycin, or 40-epi-(N1-tetrazolyl)-rapamycin), structural derivatives and functional analogues of everolimus; an mTOR inhibitor; pirfenidone; or a combination thereof. In one embodiment, the second layer may be coated with paclitaxel.

The second layer, for instance, may be coated with an anti-inflammatory agent. In one embodiment, the anti-inflammatory agent may be selected from a group consisting of novolimus, myolimus, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, montelukast, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus, or a combination thereof. In one embodiment, the second layer may be coated with montelukast.

The anti-contractile agent and the second agent may be present in the endovascular device disclosed herein at a ratio sufficient to mediate adverse arterial remodeling. For instance, the anti-contractile agent and the second agent may be present in the endovascular device at a ratio of from about 50:20 to about 20:50, such as from about 25:15 to about 15:25, such as from about 20:5 to about 5:20, or any range therebetween. In one embodiment, the anti-contractile agent and the second agent may be present in the endovascular device at a ratio of from about 10:5 to about 5:10. In one embodiment, the anti-contractile agent and the second agent may be present in the endovascular device at a ratio of about 7:10.

The drug containing layers (e.g., first layer and second layer) may also comprise urea. For instance, the anti-contractile agent, anti-inflammatory agent, or anti-proliferative agent may be dissolved in an appropriate solvent in the presence of urea and coated onto the surface of the endovascular device. The presence of urea in a drug coated layer may promote the release of the drug or agent from said layer. For instance, the presence of urea in the second layer in proximity to, or in contact with, a first layer comprising an anti-contractile agent may promote the release of the anti-contractile agent of the first layer and may promote the release of the other agent in the second layer. A suitable solvent may include, but is not limited to, tetrahydrofuran (THF), acetone, methanol, acetonitrile, dimethyl sulfoxide dioxane (DMSO), ethanol, acetone, water, or a combination thereof.

The drug coated layers (e.g., first layer and second layer) may be present in the endovascular device disclosed herein in any suitable coating composition. Further, the coating composition of the first and second layers described herein may be applied to the surface of the endovascular device in any suitable manner.

In one embodiment, the coating composition that forms the first layer may be applied to a surface of the endovascular device in any suitable manner resulting in a first layer having an anti-contractile agent, e.g., a crystalline anti-contractile agent. In one embodiment, the second layer may be applied to the surface of the endovascular device in any suitable manner resulting in a second layer having an anti-inflammatory agent, an anti-proliferative agent, or a combination thereof, e.g., a crystalline anti-inflammatory agent, anti-proliferative agent, or combination thereof.

For instance, the coating composition may be applied to the surface of the endovascular device through a variety of known techniques, such as dip, coating, brushing, spraying, microextrusion, hot-melt extrusion, injection molding, compression molding, etc. It is understood that aspects of the present disclosure are not limited to any particular application or deposition technique.

In one embodiment, spraying may be employed. Spray coating an endovascular device may involve mounting or disposing said device on a support, followed by spraying a coating composition from a nozzle onto the mounted device. Solvent is removed from the deposited coating composition to form the coating. There may be some residual solvent remaining in the coating after the solvent removal or solvent removal steps. Solvent removal may be performed through evaporation at room or ambient temperature or by heating or exposing a coated stent to a temperature above room temperature. Prior to spray coating, the surface of the endovascular device may be sterilized using ultraviolet light.

Advantageously, the endovascular device disclosed herein may treat peripheral artery disease by delivering a synergistic combination of an anti-proliferative agent and an anti-contractile agent to a subject in need thereof. Interestingly, co-delivery of an anti-proliferative agent and an anti-contractile agent to a subject in need thereof may preserve the lumen upon maladaptive inward remodeling completion. As used herein, “maladaptive remodeling” refers to, in response to a sustained stimulus (such as persistent hypertension), the process yields a homeostatic state that at the cellular level is distinct from the initial (pre-remodeling) state. In other words, the baseline local mechanical environment of mechanosensitive vascular cells is not fully restored. Whereas “adaptive remodeling” refers to the restoration of baseline values of both the flow-induced shear stress at the intima and the pressure-induced medial stress, thus restoring the local mechanical environment of vascular endothelial cells (vECs) and vascular smooth muscle cells (vSMCs), respectively.

Therapeutic effectiveness of co-delivering an anti-proliferative agent and an anti-contractile agent to a subject in need thereof may be measured based on pressure-mediated artery modeling outcomes, e.g., femoral artery remodeling outcomes, post-delivery. A predictive mathematical framework may be utilized to better understand the effects of delivered drugs on pressure-mediated artery remodeling outcomes, with the goal of simulating a post-DCB remodeling scenario in terms of the targeted vessel (e.g., femoral artery), traditional and novel payloads (anti-proliferative and anti-proliferative+anti-contractile drugs, respectively), and typical patient co-morbidities (hypertension and EC dysfunction).

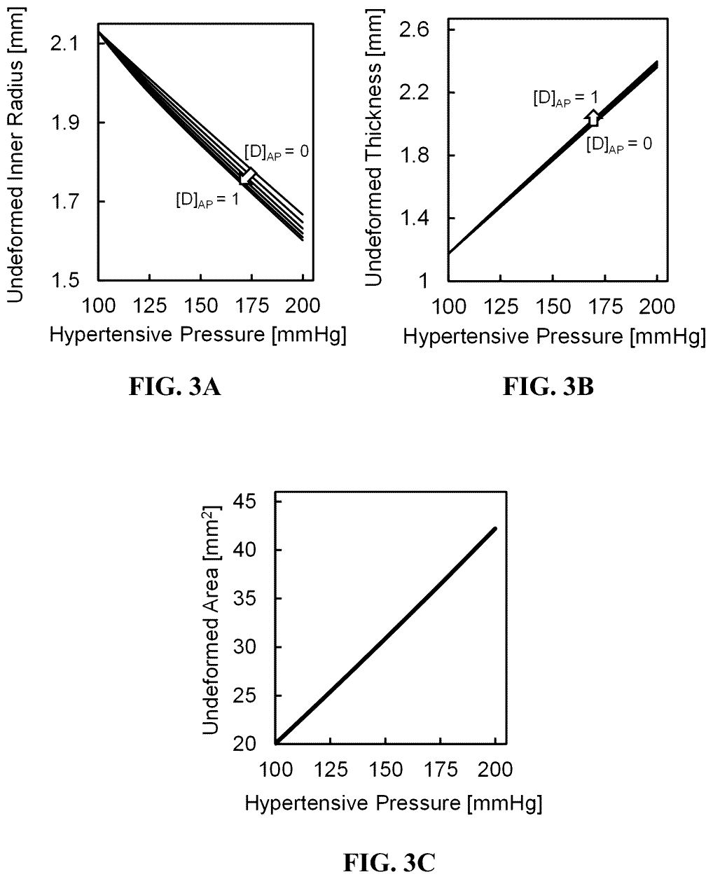

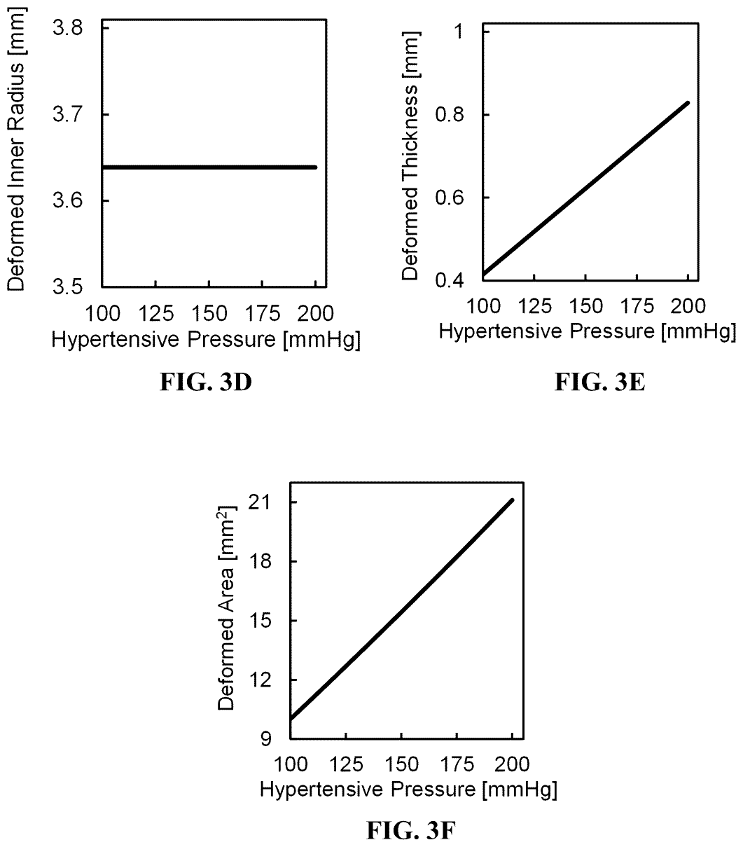

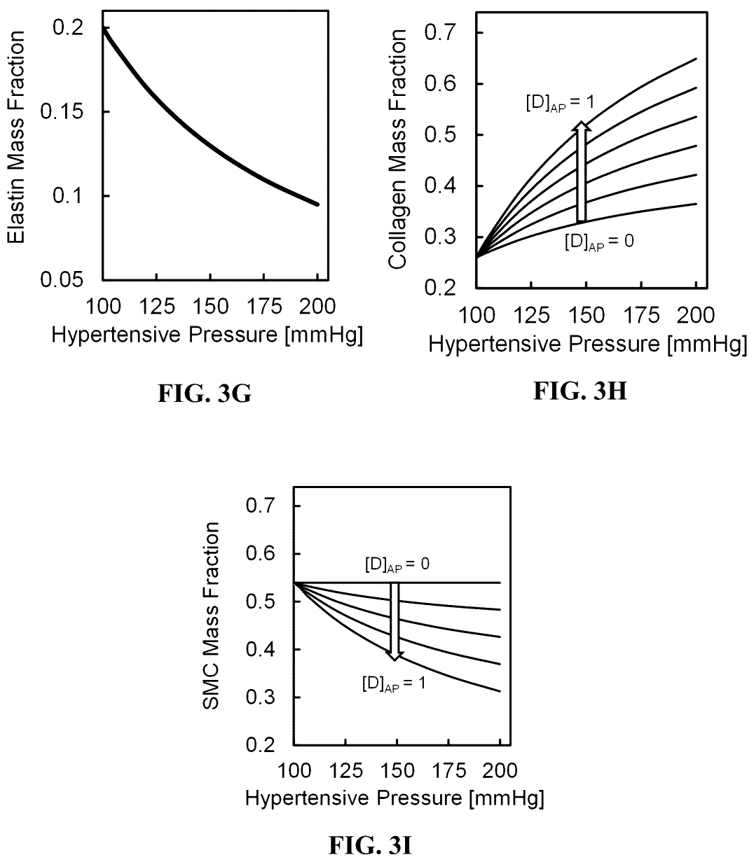

In one embodiment, a predictive adaptive pressure-mediated arterial modeling may be utilized following traditional DCB deployment to understand the effects of delivering the anti-proliferative agent and the anti-contractile agent to a subject in need thereof. Predictions of true adaptive remodeling outcomes following traditional DCB deployment show that as the degree of hypertension increases, the undeformed geometry of the remodeled vessel is characterized by a monotonic decrease in inner radius, increase in wall thickness, and increase in wall area, with moderate AP drug-mediated alterations (less than 5% over the examined range) in inner radius and wall thickness (FIGS. 3A-C). Conversely, the deformed geometry of the remodeled vessel is fully prescribed in accordance with System (10) and is therefore insensitive to AP dosing, with outward hypertrophic remodeling outcomes characterized by the retention of inner radius coupled with monotonic increases in wall thickness/area such that flow-induced shear stress and circumferential wall stress are restored to baseline values (FIGS. 3D-F). Predicted changes in wall composition commensurate with this growth include a monotonic reduction in elastin mass fraction, increase in collagen mass fraction, and retention (no AP drug) or decrease in SMC mass fraction (FIGS. 3G-I). The AP drug-mediated reduction in SMC mass (per Eq. (12)) becomes relatively more significant (in comparison to total arterial mass) with increasing hypertensive pressure, thus having a greater effect on φ₍ₐ₎^{smc} that culminates with an approximate 40% drug-mediated reduction at P(D_{[AP]}=1 vs. D_{[AP]}=0 in FIG. 31). Thus, if post-DCB remodeling is inherently adaptive, the imposed constraints on constituent mass production result in drug-mediated compositional alterations (increase in collagen content, decrease in SMC content) which could compromise the contractile function of the artery.

When considering traditional DCBs, in which an AP drug is the sole payload component, the model predicts that adaptive pressure-mediated post-DCB remodeling retains the outward hypertrophic character associated with adaptive remodeling under sustained hypertension, as well-established in theoretical, experimental, and clinical studies; AP drug dosing induces a negligible change in true remodeling outcomes and has no effect on the apparent remodeling outcomes; increasing AP drug dosing leads to a monotonic increase of collagen content and reduction of SMC content in the remodeled vessel.

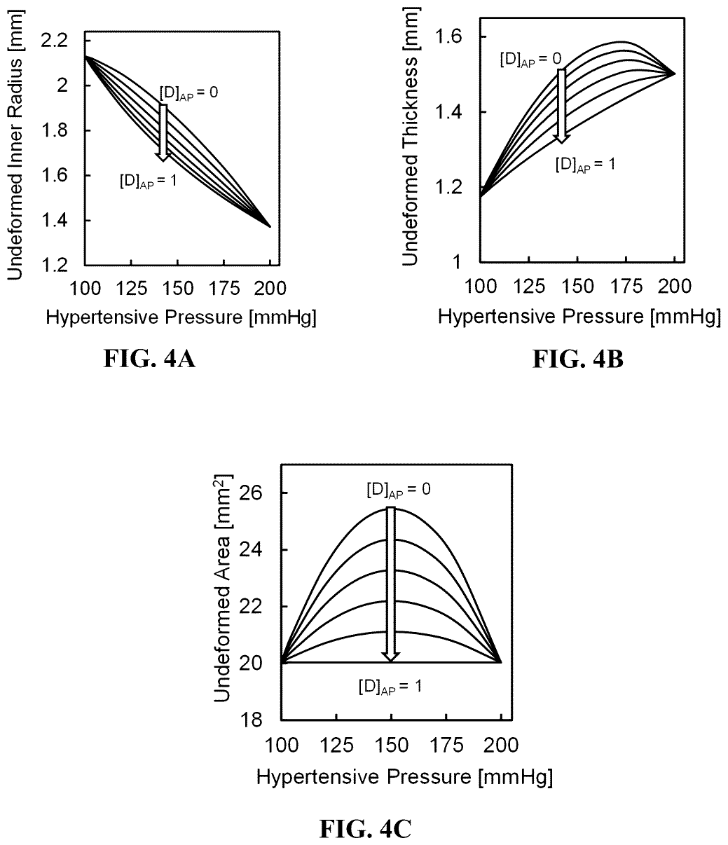

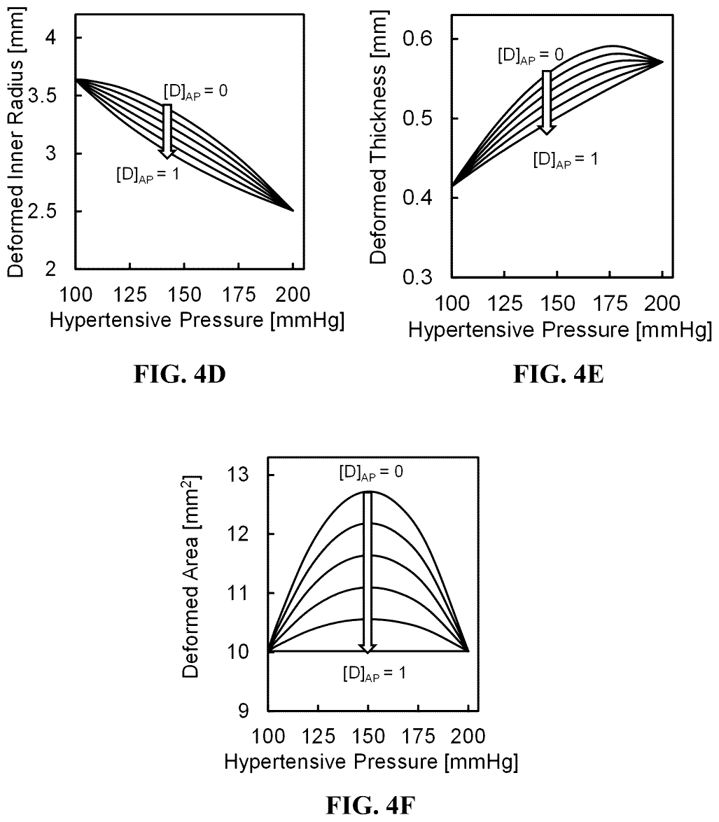

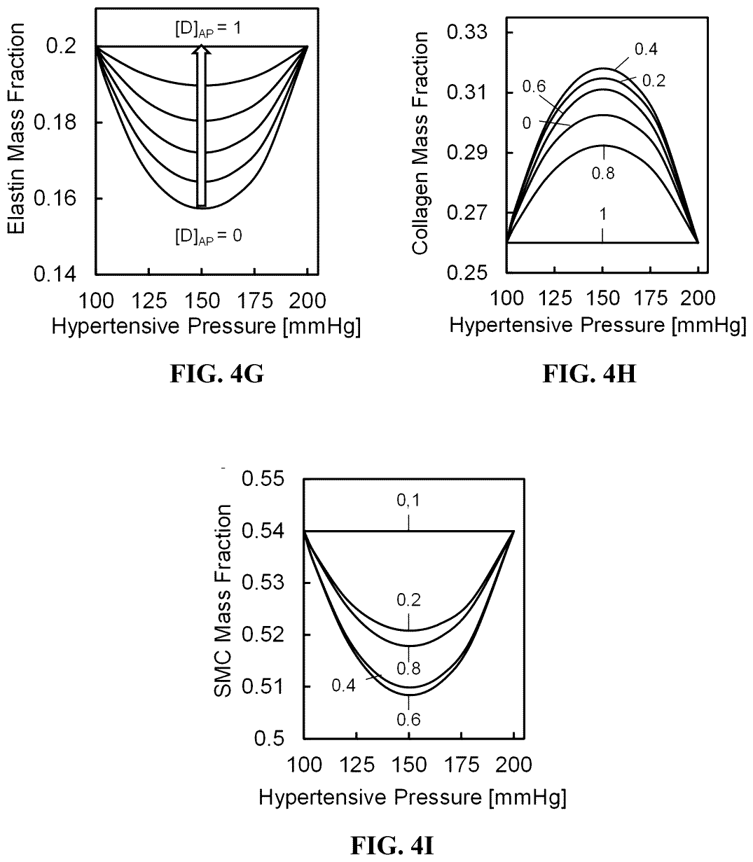

In one embodiment, a predictive maladaptive pressure-mediated arterial modeling may be utilized following traditional DCB deployment to understand the effects of delivering the anti-proliferative agent and the anti-contractile agent to a subject in need thereof. Predictions of true maladaptive remodeling outcomes following traditional DCB deployment show that as the degree of hypertension increases, there is a monotonic reduction in the undeformed inner radius, with notable AP drug-mediated enhancement of inward growth at intermediate pressures (FIG. 4A). Conversely, changes in undeformed wall thickness and area are nonmonotonic as hypertensive pressure increases, but like the inner radius these outcomes exhibit most sensitivity to AP drug at intermediate hypertension (FIGS. 4B and 4C). Predictions of deformed vessel geometry (FIGS. 4D-F) exhibited analogous trends with increasing hypertension and AP drug dosing, with apparent remodeling characterized by inward growth that transitions from hypertrophic to eutrophic as the hypertensive pressure approaches the assigned critical value as stipulated by System (15). Constituent mass fractions exhibit a complex response to AP drug dosing in this maladaptive scenario (FIGS. 4G-I), which is due to drug-mediated effects on both the SMC mass (per Eq. (12)) and total wall mass (per third Eq. in System (15)). For example, increasing D_{[AP]} over the range of [0,0.6] causes a reduction in φ₍ₘₐ₎^{smc}, whereas further increases in drug concentration causes a comparative increase in φ₍ₘₐ₎^{smc} that culminates in preservation of the baseline value when D_{[AP]}=1. Indeed, in the limiting scenario of D_{[AP]}=1, the model (per third Eq. in System (15)) enforces a full arrest of wall growth irrespective of the hypertensive pressure, and thus there is no remodeling-mediated change in tissue composition.

When considering traditional DCBs, in which an AP drug is the sole payload component, the model predicts that maladaptive pressure-mediated post-DCB remodeling is inward and hypertrophic for all hypertensive pressures below P^{cr}; AP drug diminishes remodeled arterial mass at all hypertensive pressures below P^{cr}; AP drug promotes restenosis in maladaptive inward remodeling, with the most significant drug-mediated reduction in the remodeled vessel deformed inner radius occurring at intermediate hypertension; AP drug-mediated alterations in remodeled wall composition are complex due to effects on both SMC and total mass production, but the compositional changes are overall less significant when compared to the adaptive scenario; the biphasic changes that characterize geometric remodeling outcomes under increasing degree of hypertension emerge due to pressure-dependent growth that restores the circumferential wall stress and the modeled compromise between SMC synthetic potential and tone, the latter of which is also pressure-dependent per the second equation in System (15); the biphasic nature of pressure-mediated mass fraction alterations follows from the trend in geometry coupled with the model assumption that elastin mass remains constant throughout the remodeling process.

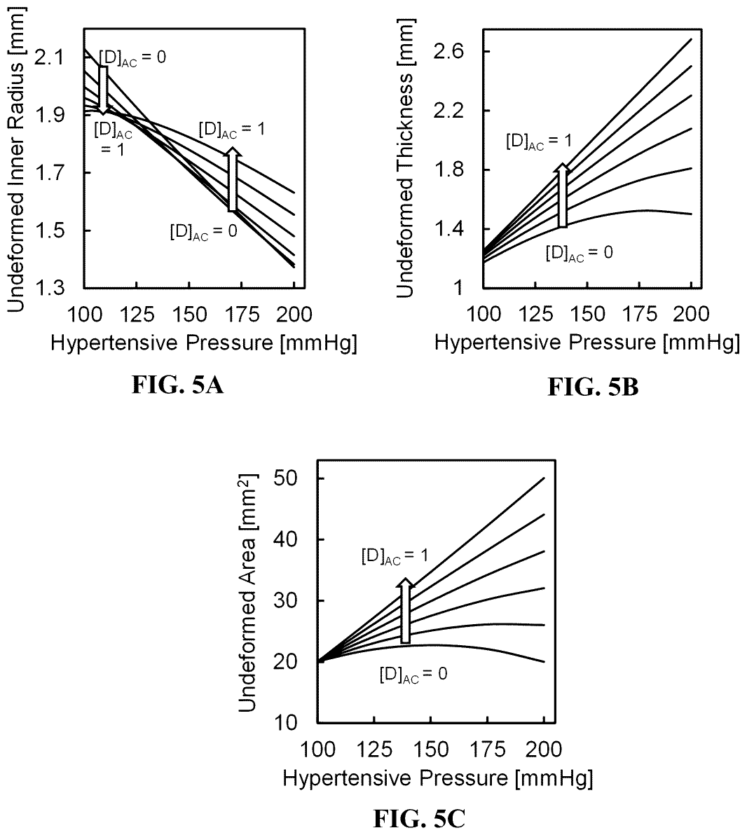

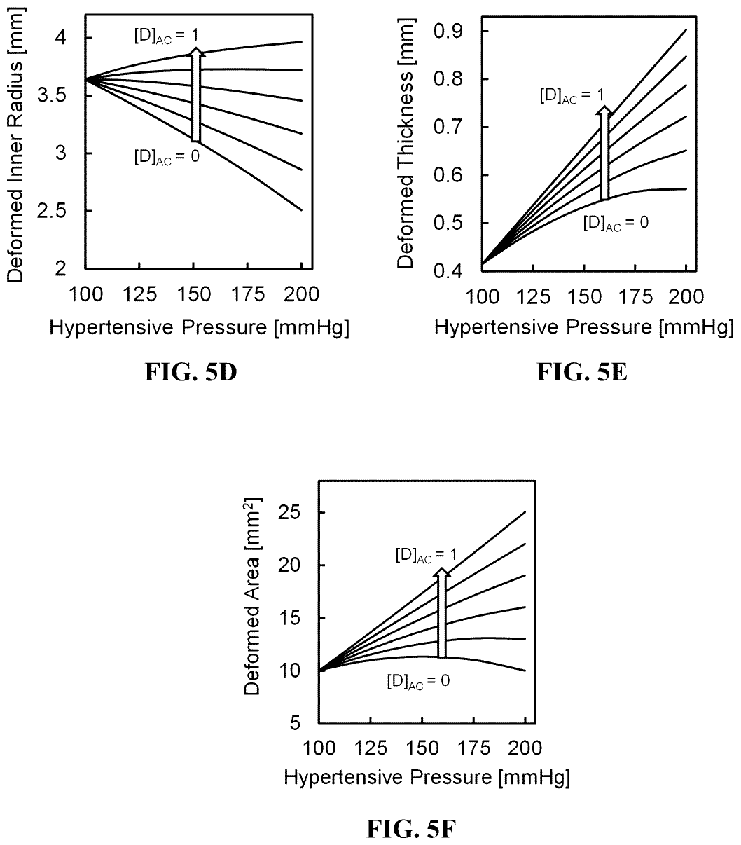

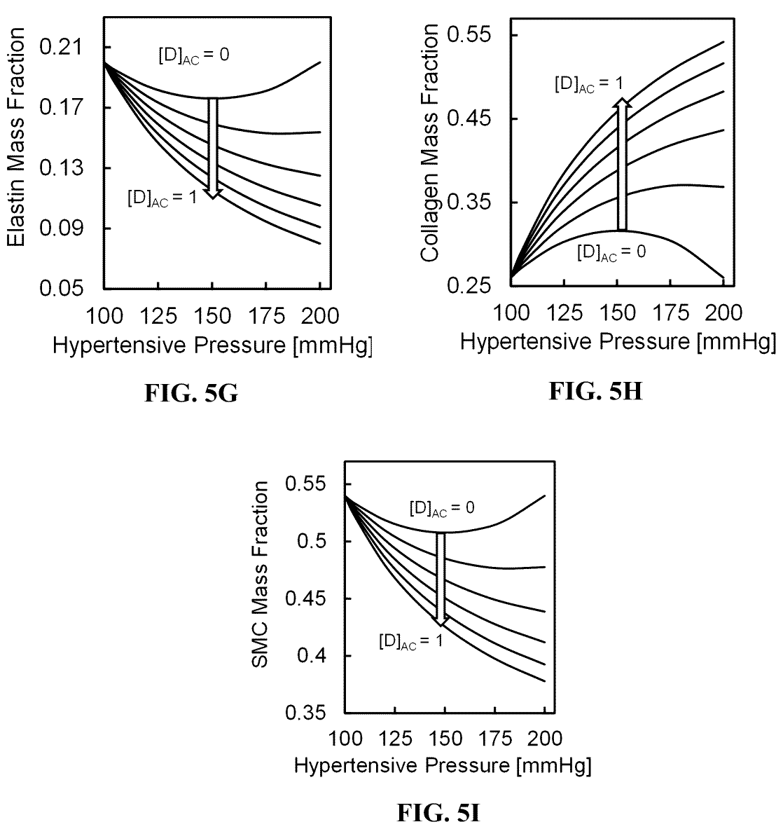

In one embodiment, a predictive maladaptive pressure-mediated arterial modeling may be utilized following novel DCB deployment to understand the effects of delivering the anti-proliferative agent and the anti-contractile agent to a subject in need thereof. Predictions of true maladaptive remodeling outcomes following novel DCB deployment show that with a fixed, intermediate dose of (traditional) AP drug, co-delivery of an AC drug significantly alters both growth direction and extent (FIGS. 5A-C). With respect to the growth direction, the AC drug promotes increasingly inward growth over the lower range of examined hypertensive pressures (P^{H}<120 mmHg), whereas at higher hypertensive pressures (P^{H}>160 mmHg) inward growth is curtailed (FIG. 5A). In terms of growth extent, increasing D_{[AP]} leads to monotonic increases in wall thickness/area for all hypertensive pressures (FIGS. 5B-C), an effect in line with the introduced tradeoff between SMC contractile/synthetic phenotypes as modeled by the third equation in System (16) along with Eq. (17). The effect of AC drug on apparent remodeling outcomes is monotonic with respect to all deformed geometrical variables, whereby increasing D_{[AP]} leads to increased inner radius, wall thickness, and wall area in all hypertensive states (FIGS. 5D-F). Critically, the model predicts an AC drug-mediated reversal of apparent growth direction (inward to outward) in all hypertensive states at the higher considered doses (FIG. 5D, D_{[AP]}=[0.8, 1]), supporting the therapeutic potential of an AC drug to mitigate restenosis in the maladaptive post-DCB context. At lower doses of AC drug, inward growth still occurs across the examined range of hypertensive pressures, suggesting that benefit to the patient would be limited by inadequate AC dosing/efficacy throughout the remodeling process. Remodeling-mediated changes in constituents' mass fraction include a decrease in φ₍ₘₐ₎^{el} and φ₍ₘₐ₎^{smc} at all hypertensive pressures (except for at D_{[AP]}=0 and P^{H}=P^{cr}, at which the response is eutrophic); for a given P^{H}, AC drug inclusion alters the ratio of wall constituents such that φ₍ₘₐ₎^{el} and φ₍ₘₐ₎^{smc} are reduced while φ₍ₘₐ₎^{col} is proportionally increased (FIGS. 5G-I).

When considering DCBs in which both AP and AC drugs are included in the payload, the model predicts that maladaptive post-DCB remodeling is significantly altered by the presence of the novel AC drug component, including changes in both true and apparent remodeling outcomes; as a consequence of the imposed tradeoff between SMC contractile and synthetic phenotypes, the AC drug promotes an increase in remodeled arterial mass at all hypertensive pressures, as well as a reversal of the growth direction from inward to outward; AC drug inclusion alters remodeled arterial wall composition, whereby drug-induced changes are monotonic across the evaluated range of hypertensive pressures and characterized by relative decreases in φ₍ₘₐ₎^{el} and φ₍ₘₐ₎^{smc} and increase in φ₍ₘₐ₎^{col}.

Implementation of the payload designs herein requires that the normalized dosing parameters, D_{[AP]} and D_{[AP]}, are registered with specific drug concentrations in novel DCBs. At a minimum, identification of these parameters for a specific drug/tissue target would require ex-vivo and/or preclinical in-vivo dosing studies that interrelate initial DCB formulation, resultant tissue concentrations (after local delivery via DCB), and the manifest SMC activity (indices of proliferation and contractility) over time scales relevant to arterial remodeling (i.e., weeks-to-months). While such studies present numerous experimental challenges, high-throughput bioreactor systems which promote tissue viability for extended periods potentiate initial specification of DCB design guided by the proposed modeling framework.

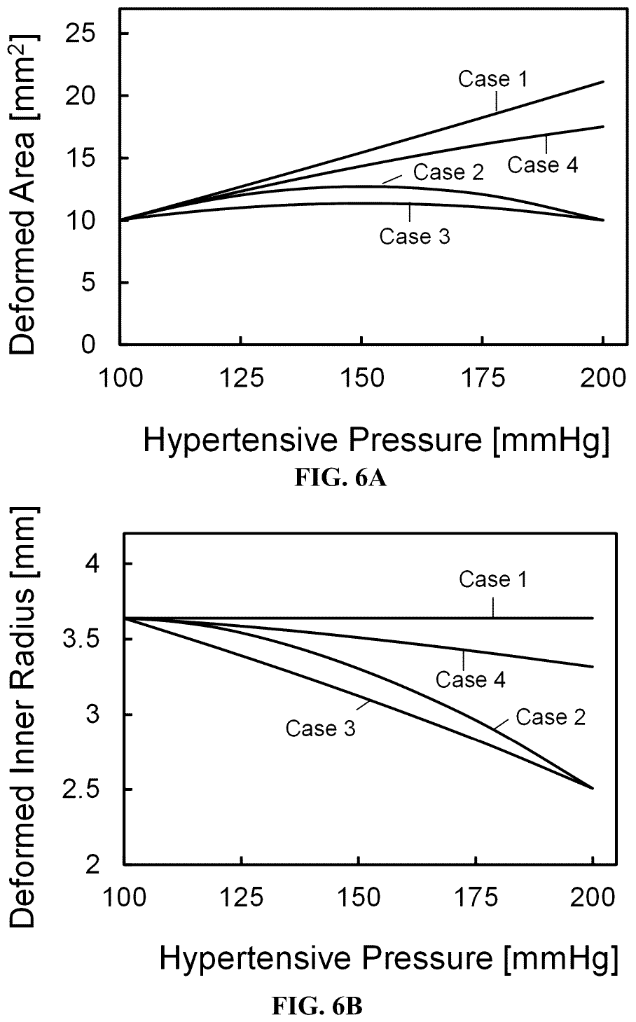

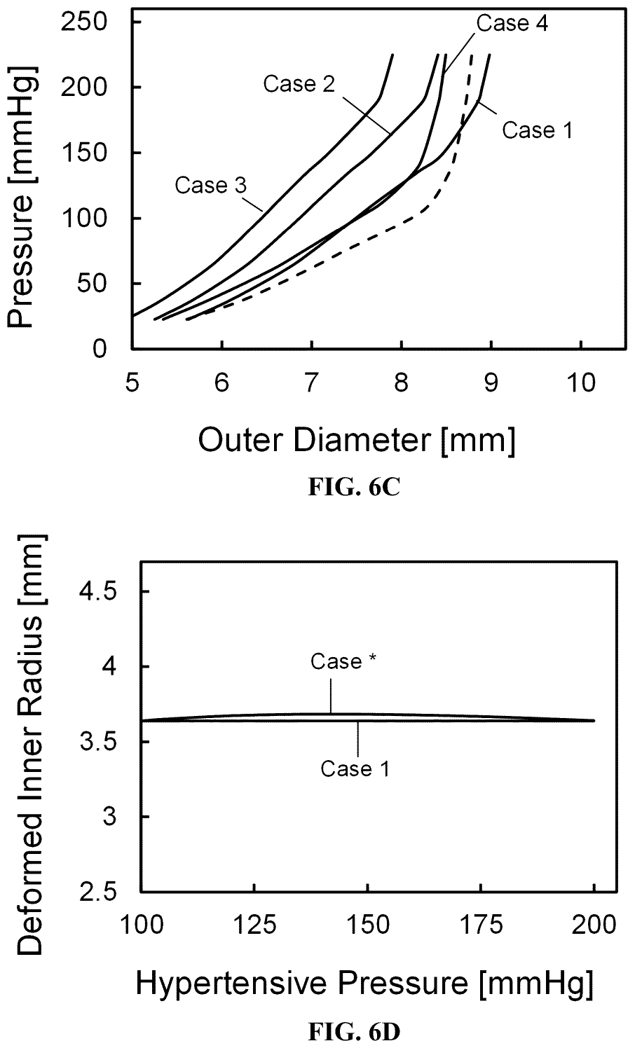

In each formulated scenarios disclosed herein for maladaptive post-DCB remodeling, the tensile circumferential stress is restored to the baseline value and as such is an adaptive outcome from the perspective of SMCs, but flow-induced shear stress at the endothelium is not restored due to EC dysfunction. Moreover, also due to EC dysfunction, we prescribe an elevation in SMC tone due to hypertension, with predicted maladaptive outcomes that align with theoretical, experimental, and clinical findings of inward growth and reduction in remodeled vessel deformability as shown in FIG. 6C. Thus, while findings herein support the potential for improving patient outcomes in terms of post-DCB lumen preservation via selective tuning of co-delivered AP and AC drugs, other outcomes of maladaptive remodeling, namely loss of vessel deformability, are predicted to by only partially mitigated by this approach.

The present disclosure may be better understood with reference to the following examples.

### EXAMPLES

### Example 1

### Materials and Methods

### Active and Passive Arterial Response.

The porcine femoral artery was first isolated from the hindquarters of a freshly slaughtered adult American Yorkshire Sow (˜3 years old; ˜200 kg) between the deep femoral bifurcation and the popliteal initiation at the popliteal fossa, whereby applied tissue marking dye was used to determine a segmental vessel length first in-situ and following isolation. The isolated vessel was immediately transported to the laboratory in Krebs saline solution on ice. Once there, perivascular tissues were gently removed, and the vessel was trimmed down to a straight section between branches roughly 4 cm in length. Next, the vessel was cannulated to hose barbed Luer fittings using 3-0 braided suture and mounted into a custom-built porcine vessel active/passive mechanical testing device. Briefly, this bi-axial testing device controls pressure and axial extension while measuring outer diameter and axial force for vessels up to 20 cm in length. The mounted vessel was continuously perfused and bathed in 37° C. temperature-controlled 7.4 pH Krebs solution aerated with purified 95% oxygen and 5% carbon dioxide from a compressed gas cylinder.

Prior to data acquisition, the vessel was preconditioned to minimize viscous effects via five cycles of axial stretching and pressurization following established protocols. To characterize the vessel response under the basal SMC state, it was extended and held at a given fixed length (equal to the corresponding in-situ length recorded prior to tissue harvest or ±5% of this length) and pressurized from 10 to 200 mmHg in increments of 10 mmHg. Here, the term “basal” reflects that SMCs are in an intermediate contractile state (between subsequently induced fully passivated and maximally contracted states) that is realized once the specimen is cannulated and acclimated within our perfusion bioreactor system. Vessel outer diameter, transmural pressure, and axial force were recorded in triplicate and averaged at each experimental state. After flushing the vessel with saline, a fully contracted SMC state was induced with an aerated bathing media containing phenylephrine (10⁻⁵ M), waited 15 minutes for tissue acclimation, and repeated the testing protocol. To assess the vessel response in the passive SMC state, we again repeated the testing protocol after flushing and adding sodium nitroprusside (10⁻⁴ M) to the bathing media.

### Arterial Wall Composition

Upon completion of mechanical testing, the arterial sample was fixed in 4% paraformaldehyde, embedded vertically in paraffin, and cut into 10 sections of 5-micrometer thickness using a microtome to obtain sequential ring-shaped profiles. All sections were deparaffinized and rehydrated in xylene and alcohol of decreasing concentrations before staining. Staining alternated between Direct Red (0.1% in saturated picric acid) for 90 minutes and mounting with Permount or Resorcin Fuchsin working solution (16 hours) with Woodstain Scarlet Acid Fuchsin solution (5 minutes) and mounting with Poly Mount fluorescent mounting media. Both staining protocols entailed dehydration in alcohol and xylene of increasing concentrations before mounting. All sections were imaged using a Zeiss Axioskop 2 microscope with either bright-field or cross-polarized light to detect collagen birefringence. ImageJ software was used to quantify collagen birefringence in darkfield images (threshold: 34-255) and total tissue area in the brightfield images (threshold: 0-210) of Direct Red-stained samples. For simplicity, it was assumed that the area fractions obtained from two-dimensional image thresholding and mass fractions were equivalent. Likewise, elastin content was measured as the ratio of black (threshold: 0-140) to all other tissue area in the Resorcin and Woodstain Scarlet Acid Fuchsin brightfield images. Neglecting ground substance, it was assumed the remaining wall constituents were predominantly circumferentially-oriented SMCs.

### Experimental Data Processing.

Obtained experimental data enabled a continuum mechanics-based analysis of the arterial response with account of the SMC contractile state. The artery is modeled as a cylindrical membrane made of an elastic, orthotropic, and incompressible material. As a result of loading (transmural pressure P and axial force f) and circumferentially-oriented SMC contraction, the vessel undergoes a finite axisymmetric deformation. The reference arterial geometry was determined by measures of passivated/unloaded sample length (L), inner radius (R i), and wall thickness (H), which were used to compute wall volume (V) as

V=π((Rᵢ+H)²−Rᵢ²)L. (1)

The incompressibility assumption allows calculation of the vessel inner radius rᵢ at each deformed state, namely

$${r}_{i} = \sqrt{{r}_{o}^{2} - \frac{V}{π l}} \qquad ( 2 )$$

where rₒ and l are the deformed outer radius and length, respectively.

The deformed state is described by the deformation of the mid-wall arterial surface in terms of the right Cauchy-Green strain tensor C=diag{λ₁², λ₂²} and the change in wall thickness. The principal stretches in the axial (λ₁) and circumferential (λ₂) direction and the deformed wall thickness (h) are given by

$${λ}_{1} = \frac{l}{L} \qquad ( 3 )$$

$${λ}_{2} = \frac{{r}_{i} + h / 2}{{R}_{i} + H / 2}$$

$$h = \frac{H}{{λ}_{1} {λ}_{2}} .$$

Eq. (2) in combination with recorded experimental data enable calculation of mean principal wall stresses in the axial (σ₁) and circumferential (σ₂) direction at each deformed state:

$${σ}_{1} = \frac{f + P π {r}_{i}^{2}}{π h ( 2 {r}_{i} + h )} \qquad ( 4 )$$

$${σ}_{2} = \frac{P {r}_{i}}{h} .$$

The tissue was considered as a constrained mixture of two passive load-bearing constituents (elastin and collagen) and one active constituent (SMCs), hereafter denoted by superscripts el, col, and smc, respectively. The mass fractions (φ) of each constituent are:

$${φ}^{e l} = \frac{{M}^{e l}}{M} \qquad ( 5 )$$

$${φ}^{col} = \frac{{M}^{c o l}}{M}$$

$${φ}^{smc} = \frac{{M}^{s m c}}{M} ,$$

where {M^{el}, M^{col}, M^{smc}} are the masses of each constituent; M is the total tissue mass.

Consideration of only these three tissue constituents implies

φ^{el}+φ^{col}+φ^{smc}=1 and M^{el}+M^{col}+M^{smc}=M. (6)

The stress field in the arterial wall is described as a sum of passive Cauchy stress tensor, σ^{pas}=diag{σ₁^{pas}, σ₂^{pas}}, and an active circumferential stress σ₂^{act}. Given the passive strain energy function (W(C)) and a general form for σ₂^{act}, the total arterial wall stresses are

$${σ}_{1} = {λ}_{1} \frac{\partial W}{\partial {λ}_{1}} , \qquad ( 8 )$$

$${σ}_{2} = {λ}_{2} \frac{\partial W}{\partial {λ}_{2}} + {σ}_{2}^{act} = {λ}_{2} \frac{\partial W}{\partial {λ}_{2}} + {φ}^{s m c} S F ( {λ}_{2} ) .$$

As shown in the second equation, the magnitude of σ₂^{act} depends on the SMC content (via φ^{smc}), activation state (via activation parameter S), and deformation (via normalized function F (λ₂)).

The flow-induced shear stress at the endothelium is τ=4 μQ/ρrᵢ³, where Q is the volumetric flow rate and μ is the dynamic blood viscosity.

The equation of equilibrium in the radial direction that follows from a free-body diagram of a vessel inflated by an internal pressure is σ₂=Prᵢ/h, often called Laplace's law, can be expressed as

$${λ}_{2} \frac{\partial W}{\partial {λ}_{2}} + {φ}^{s m c} S F ( {λ}_{2} ) = P \frac{[ {λ}_{1} {λ}_{2}^{2} ( {R}_{i} + \frac{H}{2} ) \frac{H}{2} ]}{H} . \qquad ( 9 )$$

Given W(C), initial dimensions Rᵢ and H, axial stretch λ₁, constituent mass fractions, activation parameter S, lumen pressure P, and the function F(λ₂), Eq. (9) enables calculation of the circumferential stretch λ₂ when the vessel is in equilibrium.

When an artery is inflated by the normotensive pressure P^{N}, extended to in-situ axial stretch λ₁=λ, and SMCs are under a basal level of stimulation, the corresponding strain and stress values are termed as basal and are hereafter denoted by the subscript (b).

### Remodeling Scenario 1: Adaptive Pressure-Mediated Arterial Remodeling Following Traditional DCB Deployment.

The global growth approach was utilized to predict the outcomes of pressure-mediated arterial remodeling, which relies on formulate and solve equation systems that reflect observed/plausible characteristics of the remodeling process. While this study focuses on maladaptive pressure-mediated post-DCB remodeling, the framework herein entails specification of adaptive remodeling outcomes under equivalent hypertensive pressures (as explained below). Thus, outcomes were initially used to predict outcomes of adaptive pressure-mediated post-DCB remodeling to both inform the proposed model of maladaptive remodeling and provide context for the obtained results herein.

Upon completion of adaptive post-DCB remodeling under a hypertensive pressure P^{H}, the vessel is assumed to manifest an increase in wall thickness with no change in inner radius—as a result of these geometrical changes, remodeling restores basal values of flow-induced shear stress (assuming no change in flow rate) and circumferential wall stress. Additionally, it is assumed that constituent mechanical properties, SMC contractile state, and axial stretch are not altered by an adaptive remodeling process.

In line with these assumptions, we formulate and solve the following system of governing equations to calculate the geometrical parameters that describe apparent growth due to adaptive remodeling, i.e., rᵢ₍ₐ₎ and h₍ₐ₎, with the subscript (a) indicating an adaptive remodeling outcome:

$${r}_{i ( a )} = {r}_{i ( b )} {h}_{( a )} = {h}_{( b )} \frac{{P}^{H}}{{P}^{N}} . \qquad ( 10 )$$

The first equation in System (10) enforces the restoration of flow-induced shear stress; the second equation enforces the restoration of circumferential wall stress.

System (10) equations can be combined to express the deformed cross-sectional wall area of the remodeled artery (a₍ₐ₎) as

$${a}_{( a )} = 2 π ( {r}_{i ( b )} + \frac{{h}_{( b )}}{2} \frac{{P}^{H}}{{P}^{N}} ) {h}_{( b )} \frac{{P}^{H}}{{P}^{N}} \qquad ( 11 )$$

It was assumed that in the absence of an anti-proliferative (AP) drug, pressure-mediated adaptive remodeling results in an increase in SMC mass that is proportional to the increase in total arterial mass such that φ^{smc} remains constant, reflecting a partial condition imposed in previous models of adaptive arterial remodeling in hypertension. To model the effect of an AP drug on resident SMCs in a manner in-line

with in-vitro and in-vivo findings [18], the following relation in which drug dosing limits SMC mass increase during the remodeling process was introduced:

$${M}_{( a )}^{s m c} = {M}_{( b )}^{s m c} [ \frac{{a}_{( a )}}{{a}_{( b )}} + {D}_{[ A P ]} ( 1 - \frac{{a}_{( a )}}{{a}_{( b )}} ) ] . \qquad ( 12 )$$

The normalized drug dosing parameter (D_{[AP]}) ranges from 0 to 1, where in these limits the increase in SMC mass in the remodeled artery is proportional to wall growth (D_{[AP]}=0) or fully arrested (D_{[AP]}=1).

The mass of elastin is assumed to be unaltered by the remodeling process, which has been experimentally observed and understood to be a consequence of the exceedingly low turn-over rate of this protein, but the mass fraction changes due to changes in other constituents' mass. Thus, with these assumptions and making use of Systems (5) and (6), constituent mass fractions in the remodeled artery are:

$${φ}_{( a )}^{e l} = \frac{{M}_{( b )}^{e l}}{{M}_{( a )}} \qquad ( 13 )$$

$${φ}_{( a )}^{c o l} = 1 - {φ}_{( a )}^{e l} - {φ}_{( a )}^{s m c}$$

$${φ}_{( a )}^{s m c} = \frac{{M}_{( a )}^{s m c}}{{M}_{( a )}} .$$

Finally, to calculate the geometrical parameters that describe true growth due to adaptive pressure-mediated remodeling, i.e. Rᵢ₍ₐ₎ and H₍ₐ₎, and deformation parameter λ₂₍ₐ₎, the following system was utilized:

$${λ}_{2 ( a )} ( {R}_{i ( a )} + \frac{{H}_{( a )}}{2} ) - \frac{{H}_{( a )}}{2 λ {λ}_{2 ( a )}} - {r}_{i ( b )} = 0 \qquad ( 14 )$$

$$\frac{{H}_{( a )}}{λ {λ}_{2 ( a )}} - {h}_{( b )} \frac{{P}^{H}}{{P}^{N}} = 0$$

$${λ}_{2 ( a )} \frac{\partial W}{\partial {λ}_{2 ( a )}} + {φ}_{( b )}^{s m c} {S}_{( b )} F ( {λ}_{2 ( a )} ) - {σ}_{2 ( b )} = 0.$$

The first and second equation in System (14) are analogous to System (10) but expressed in terms of the undeformed vessel dimensions, while the third equation incorporates the constitutive equation into the condition that remodeling restores the circumferential wall stress.

### Remodeling Scenario 2: Maladaptive Pressure-Mediated Arterial Remodeling Following Traditional DCB Deployment.

Here, post-DCB femoral artery remodeling was considered in response to a sustained hypertensive pressure P^{H} as a maladaptive process due to EC dysfunction, which compromises EC-mediated regulation of SMC contractility. Previous studies suggest that maladaptive inward remodeling in hypertension is associated with EC dysfunction and an increase in SMC tone—the predominance of the contractile phenotype consequently diminishes the upregulation of matrix synthesis as seen in adaptive remodeling. In addition to this tradeoff, the AP drug effects on SMCs were accounted for via imposed conditions on remodeling-mediated changes in both arterial tissue composition as well as total mass. It was assumed that basal values of the axial stretch and flow are unaffected by remodeling.

In line with these notions, our mathematical model of post-DCB maladaptive remodeling assumes the following: (i) EC dysfunction causes a pressure-mediated elevation in SMC contraction, (ii) remodeled arterial mass is reduced as compared to the case of adaptive remodeling under equivalent hypertensive pressures, with the extent of reduction dependent on both the increase in SMC contraction (due to EC dysfunction) and the decrease in SMC proliferation (due to the AP drug), and (iii) the circumferential wall stress is restored to its basal value as in the case of adaptive remodeling. Hereafter, terms labeled with the subscript (ma) refer to outcomes of maladaptive post-DCB remodeling.

To calculate the geometrical parameters that describe apparent growth due to post-DCB maladaptive remodeling, i.e. rᵢ₍ₘₐ₎ and h₍ₘₐ₎, the following system of governing equations was utilized:

$$\frac{{P}^{H} {r}_{i ( m a )}}{{h}_{( m a )}} - {σ}_{2 ( b )} = 0 \qquad ( 15 )$$

$${S}_{( m a )} = \{ \begin{matrix}{S}_{( b )} + ( {S}_{( max )} - {S}_{( b )} ) \frac{{P}^{H} - {P}^{N}}{{P}^{c r} - {P}^{N}} & if {P}^{H} \leq {P}^{cr} \\ {S}_{( max )} & if {P}^{H} > {P}^{cr}\end{matrix}$$

$$2 π ( {r}_{i ( ma )} + \frac{{h}_{( m a )}}{2} ) {h}_{( m a )} - {a}_{( b )} + ( \frac{{S}_{( max )} - {S}_{( m a )}}{{S}_{( max )} - {S}_{( b )}} ) ( 1 - {D}_{[ A P ]} ) ( {a}_{( b )} - {a}_{( a )} ) = 0 .$$

The first equation in System (15) reflects the restoration of the baseline circumferential stress; the second equation gives the relationship between hypertensive pressure P^{H} and the value of the activation parameter S∈(S_{(b)},S₍ₘₐₓ₎), where S₍ₘₐₓ₎ reflects the maximal contractile capacity of SMCs and is assigned for pressures equal or higher than a certain critical value P^{cr}; the third equation relates remodeling-mediated growth variables to SMC contractility and AP drug dosing, and insomuch reflects a trade-off among the synthetic, contractile, and proliferative phenotypes of the resident cell population. The third equation stipulates that the deformed wall area after maladaptive remodeling under a prescribed level of hypertension is decreased relative to the adaptive response wall area (at the same level of hypertension), with the extent of decrease dependent on the degree of SMC stimulation and AP drug dosing.

AP drug dosing is assumed to impact wall composition in the same manner as in the adaptive scenario and is thus given by Eqs. (12)-(13) with replacement of subscript (a) with (ma) for all terms.

Finally, as done in the previous subsection, to calculate the true growth, the first and the third equations in System (15) are rewritten in terms of the initial arterial dimensions and circumferential stretch λ₂₍ₘₐ₎, and the expression for restoration of the circumferential stress σ₂₍ₘₐ₎ is given as a sum of passive and active stress. The set of governing equations becomes

$${P}^{H} \frac{[ λ {λ}_{2 ( m a )}^{2} ( {R}_{i ( m a )} + \frac{{H}_{( m a )}}{2} ) - \frac{{H}_{( m a )}}{2} ]}{{H}_{( m a )}} - {σ}_{2 ( b )} = 0 \qquad ( 16 )$$

$${S}_{( m a )} = \{ \begin{matrix}{S}_{( b )} + ( {S}_{( max )} - {S}_{( b )} ) \frac{{P}^{H} - {P}^{N}}{{P}^{c r} - {P}^{N}} & if {P}^{H} \leq {P}^{cr} \\ {S}_{( max )} & if {P}^{H} > {P}^{cr}\end{matrix}$$

$$2 π [ {λ}_{2 ( m a )} ( {R}_{i ( ma )} + \frac{{H}_{( m a )}}{2} ) - \frac{{H}_{( m a )}}{2 λ {λ}_{2 ( m a )}} ] \frac{{H}_{( m a )}}{2 λ {λ}_{2 ( m a )}} - {a}_{( b )} + ( \frac{{S}_{( max )} - {S}_{( m a )}}{{S}_{( max )} - {S}_{( b )}} ) ( 1 - {D}_{[ A P ]} ) ( {a}_{( b )} - {a}_{( a )} ) = 0$$

$${λ}_{2 ( m a )} \frac{\partial W}{\partial {λ}_{2 ( m a )}} + {φ}_{( ma )}^{s m c} {S}_{( ma )} F ( {λ}_{2 ( m a )} ) - {σ}_{2 ( b )} = 0.$$

### Remodeling Scenario 3: Maladaptive Pressure-Mediated Arterial Remodeling Following Novel DCB Deployment.

Although current DCBs exclusively deliver AP drugs as the bioactive payload component, the modeling framework was extended to predict the effects of anti-contractile (AC) drug co-delivery on maladaptive post-DCB remodeling outcomes. To account for a non-specified AC drug, it was assumed that the SMC activation parameter S₍ₘₐ₎ remains dependent on the hypertensive pressure P^{H}, but is also modulated by the AC drug dose within the DCB payload. Like for the AP drug above, a normalized AC dosing parameter (D_{[AP]}) was introduced that ranges from 0 to 1, wherein these limits the SMC activation parameter in the remodeled artery is as defined for the maladaptive scenario (D_{[AP]}=0) or valued at zero to reflect fully passivated SMCs (D_{[AP]}=1). Specifically,

$$( 17 )$$

$${S}_{( m a )} = \{ \begin{matrix}\{ {S}_{( b )} + ( {S}_{( max )} - {S}_{( b )} ) \frac{{P}^{H} - {P}^{N}}{{P}^{c r} - {P}^{N}} \} ( 1 - {D}_{[ A C ]} ) & if {P}^{H} \leq {P}^{cr} \\ {S}_{( max )} ( 1 - {D}_{[ A C ]} ) & if {P}^{H} > {P}^{cr}\end{matrix}$$

After replacing the second equations in Systems (15) and (16) with Eq. (17), the resultant systems were used to compute both apparent and true growth upon completion of maladaptive remodeling following novel DCB deployment.

### Results

### Mechanical Response

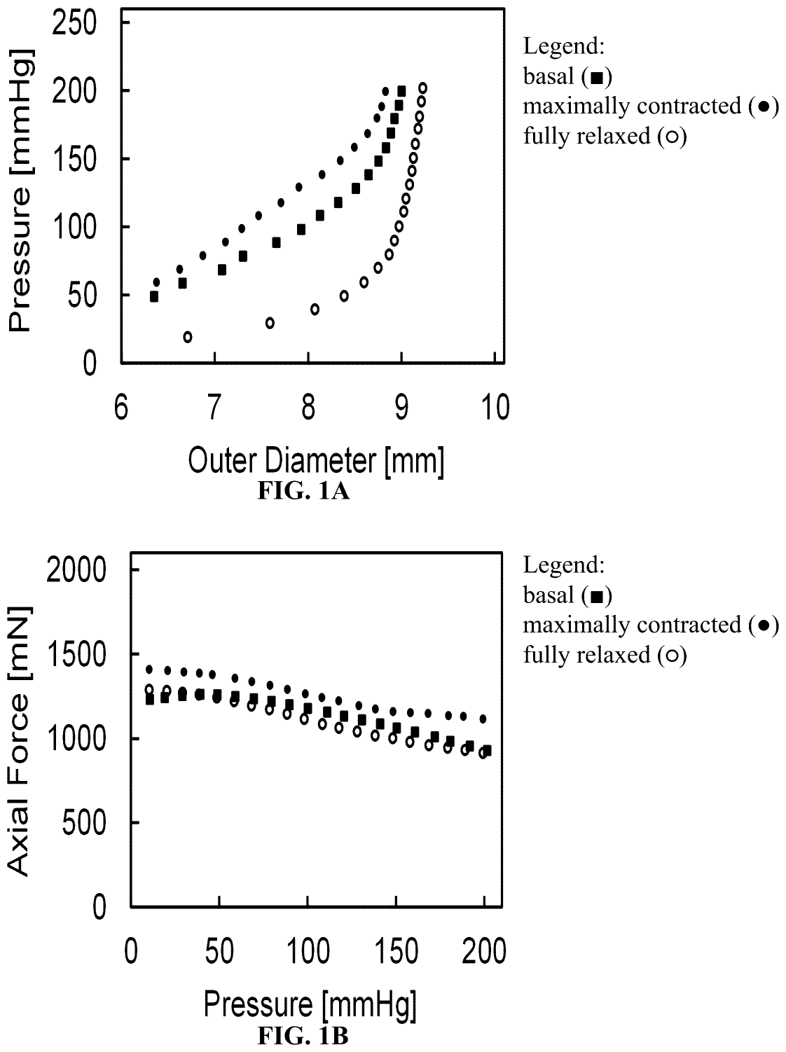

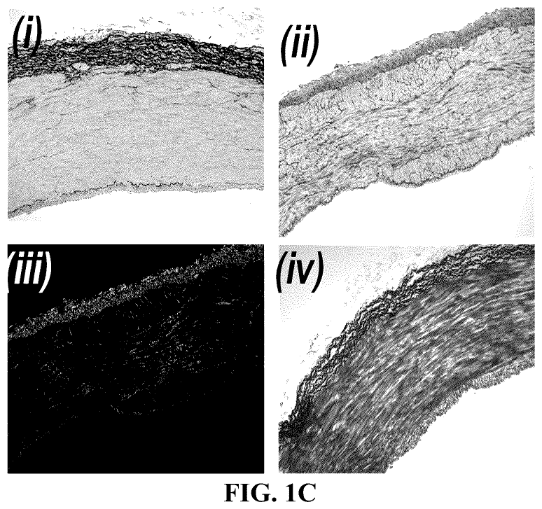

The femoral artery exhibited a nonlinear pressure-diameter response (at constant axial stretch) that is typical for muscular arteries, wherein biochemical induction of SMC contraction/relaxation induced a leftward/rightward shifting of the response curve with respect to the basal state (FIG. 1A). The axial force-pressure response (FIG. 1B) also showed predicted trends and modulation via SMC contractile state. At basal stimulation and axial stretch of 2.0, the axial force-pressure response was relatively constant, and as such the in-situ axial stretch λ was assigned this value. Analogous pressure-diameter and force-pressure data obtained at λ₁=1.9 and λ₁=2.1 showed qualitatively similar trends and were used for material property identification (not shown).

Histological images (FIG. 1C) were processed to identify constituent mass fractions for elastin, collagen, and SMCs, with obtained values φ^{el}=0.20, φ^{col}=0.26, and φ^{SMC}=0.54 reflective of a typical muscular artery.

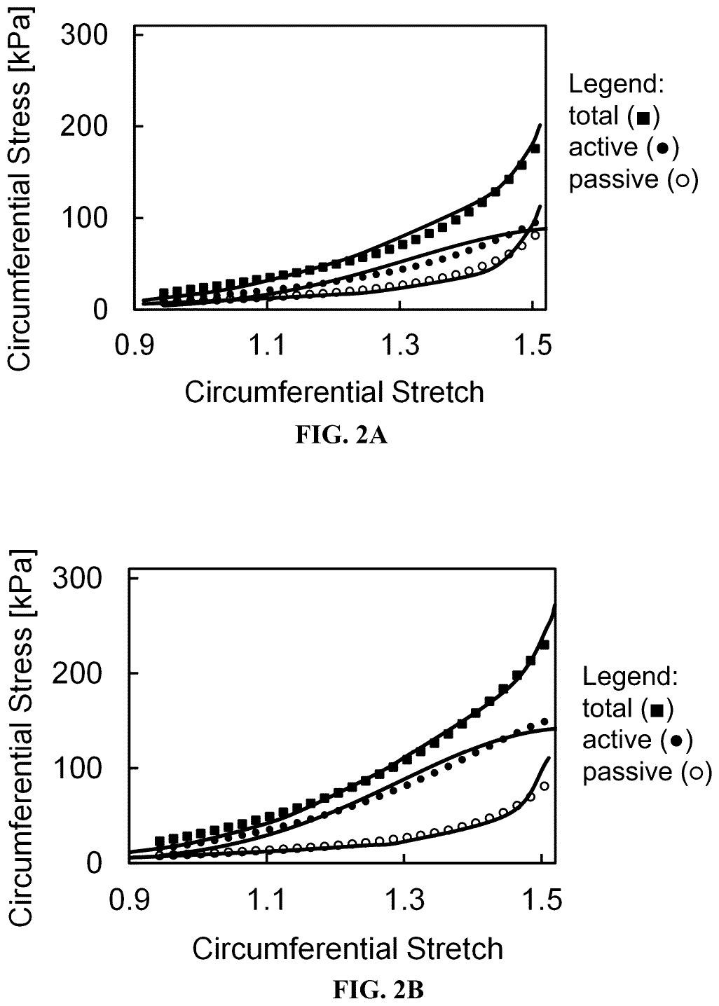

Isometric comparison between response curves under contracted (basal and maximal SMC contraction) and relaxed (passive) SMC states were used to generate total, passive, and active circumferential stretch-circumferential stress relations (FIG. 2, data points), where again responses typical of muscular arteries were observed.

To model the passive properties of the arterial tissue, we incorporated constituent mass fractions into a four-fiber family strain energy function (W) as follows:

$$W = {φ}^{e l} \frac{c}{2} ( {I}_{1} - 3 ) + {\sum }_{k = 1 …4} {φ}^{col , k} \frac{{c}_{1}^{k}}{4 {c}_{2}^{k}} \{ exp [ {{c}_{2}^{k} ( {( {λ}^{k} )}^{2} - 1 )}^{2} ] - 1 \} , \qquad ( 18 )$$

where the first term accounts for the elastin-dominated isotropic matrix that is modeled by material parameter c; the second term (summation) accounts for collagen fiber families aligned in the axial (k=1), circumferential (k=2), and diagonal (k=3 and 4) directions; fiber families are modeled by material parameters c₁^{k} and c₂^{k}, with symmetric orientation and equivalent properties for diagonal fiber families; total collagen content φ^{col} is assumed to be equally distributed among fiber families, i.e

$${φ}^{col , k} = \frac{{φ}^{c o l}}{4} for k = 1 … 4 ;$$

l₁ is the first invariant of the right Cauchy-Green strain tensor; λₖ is the stretch experienced by the k th fiber family, and is given by λ^{k}=\sqrt{λ₁² cos¹α₀^{k}+λ₂² sin²α₀^{k}} where α₀^{k} is the angle of orientation between the k^{th} fiber family and the axial direction.

Passive model parameters were identified via nonlinear regression with mechanical response data obtained under the passivated SMC state, wherein the following error function was minimized:

$$error = {\sum }_{j = 1}^{n} [ {( \frac{{P}^{m o d} - {P}^{exp}}{\overline{{P}^{exp}}} )}_{j}^{2} + {( \frac{{F}^{m o d} - {F}^{exp}}{\overline{{F}^{exp}}} )}_{j}^{2} ] . \qquad ( 19 )$$

with n the total number of j observations of measured transmural pressure or axial force in the dataset with an overbar denoting the mean values. The experimental data (exp) was measured directly while model (mod) values of axial force or pressure were calculated via equations given in Systems (4) and (8). The multivariate regression analysis was performed using the lsqnonlin function within MATLAB's optimization toolbox (MathWorks—Natick, MA) with the root mean square error (RMSE) reported.

To model the active properties, a previously used constitutive equation was adopted for the active circumferential stress, where

$${σ}_{2}^{act} = {φ}^{s m c} S {e}^{- {b [ \frac{{λ}_{M} - {λ}_{2}}{{λ}_{M}} ]}^{2}} . \qquad ( 20 )$$

The active stress is thus determined by the SMC amount/activation and a normalized function of the tissue stretch via dimensionless parameters b and λ_{M}, where the latter is the circumferential stretch for which σ₂^{act} is maximal.

Active model parameters were identified via nonlinear regression with mechanical response data obtained under activated SMC states (basal and maximal contraction), wherein the following error function was minimized:

$$error = {\sum }_{j = 1}^{n} {( \frac{{( {σ}_{2}^{a c t} )}^{m o d} - {( {σ}_{2}^{a c t} )}^{exp}}{< {( {σ}_{2}^{a c t} )}^{exp} >} )}_{j}^{2} \qquad ( 21 )$$

where n is the total number of j observations of active circumferential stress in basal and maximally contracted data sets and < > denotes the mean value. The experimental active stress (exp) was determined from isometric comparisons between the contracted and fully passivated response curves, while model (mod) values were calculated via Eq. (20). In the regression scheme, model parameters b and λ_{M} were held constant between contracted SMC states, while S varied to yield values reflective of basal (S_{(b)}) and maximal (S₍ₘₐₓ₎) SMC contraction. The multivariate regression analysis was performed using the lsqnonlin function within MATLAB's optimization toolbox (MathWorks—Natick, MA).

The measured vessel reference geometry, composition, identified values for passive/active model parameters, and RMSE are given in Table 1. Theoretically determined circumferential stress-stretch relations showed excellent agreement with the experimental data in both the basal and maximally contracted SMC states (FIG. 2, curves).

### Illustrative Simulations

TABLE 1
Geometry, wall composition, and identified constitutive
properties of the porcine femoral artery. Root mean
square error (RMSE) of theoretical versus experimental
bi-axial passive and active mechanical response.
Geometry and Composition

| | |
|---|---|
| Rᵢ (mm) | 2.131 |
| H (mm) | 1.173 |
| φ^{el} | 0.20 |
| φ^{col} | 0.26 |
| φ^{smc} | 0.54 |

Passive properties

| | |
|---|---|
| c (kpa) | 56.80 |
| c₁¹ (kpa) | 35.84 |
| c₁² (kpa) | 1.908 |
| c₁³ = c₁⁴ (kpa) | 8.231 |
| c₂¹ | 0.006 |
| c₂² | 3.082 |
| c₂³ = c₂⁴ | 0.972 |
| α₀³ = −α₀⁴ (deg) | 59.26 |
| RMSE | 0.140 |

Active properties

| | |
|---|---|
| b | 19.08 |
| λ_{M} | 1.545 |
| S_{(b)} (kpa) | 166.4 |
| S₍ₘₐₓ₎ (kpa) | 263.7 |
| RMSE | 0.466 |

The commercial numerical solver Maple 12 (Maplesoft™, Waterloo, ON, Canada) was first used to solve the governing equations referring to the vessel basal state, defined by the initial wall composition, normotensive pressure P^{N}=100 mmHg, in-situ axial stretch λ=2.0, and SMC activation parameter S_{(b)}=166.4 kPa, which yielded a deformed geometry r_{i(b)}=3.64 mm, h_{(b)}=0.41 mm, and a_{(b)}=10.02 mm² and circumferential wall stress σ_{2(b)}=117.1 kPa. Next, the solver was used to obtain numeric solutions for the governing equations in each of the post-DCB remodeling scenarios, with consideration of fixed axial stretch λ=2.0 and P^{H} ranging from 100 mmHg to 200 mmHg in 10 mmHg increments in all scenarios. In remodeling scenarios 1 and 2, the AP drug dosing parameter D_{[AP]} ranged from 0 to 1 in increments of 0.2; in remodeling scenario 3, D_{[AP]} was fixed at 0.5, while the AC drug dosing parameter D_{[AP]} ranged from 0 to 1 in increments of 0.2. In remodeling scenarios 2 and 3, the assigned critical pressure was P^{cr}=200 mmHg. For all scenarios, obtained solutions for the undeformed/deformed vessel geometry and wall composition upon remodeling completion are depicted as a function of the hypertensive pressure at the specified drug dosing (FIGS. 3-5).

Herein, a direct comparison of the following cases was done to highlight the implications of DCB therapy: Case 1—adaptive pressure-mediated arterial remodeling (no delivered drug); Case 2—maladaptive pressure-mediated arterial remodeling (no delivered drug); Case 3—maladaptive pressure-mediated post-DCB arterial remodeling with an intermediate AP drug dose of (D_{[AP]}=0.5); Case 4—maladaptive pressure-mediated post-DCB arterial remodeling, with intermediate doses of AP and AC drugs (D_{[AP]}=0.5 and D_{[AP]}=0.5). Comparison of apparent geometric outcomes of remodeling (FIGS. 6A and 6B) shows that in maladaptive cases in which AP drug is delivered alone, it decreases wall growth but promotes an inward response (Case 2 vs Case 3). Thus, these results suggest that in clinical scenarios of DCB deployment in which EC dysfunction and hypertension present as co-morbidities, an AP drug such as paclitaxel will not prevent restenosis, but instead its occurrence will be determined by the degree to which EC dysfunction impacts the post-DCB remodeling process. When DCB therapy is augmented via co-delivery of an AC drug, pressure-mediated trends in maladaptive remodeling outcomes shift towards the adaptive outcomes (FIGS. 6A and 6B, Case 4 vs Case 1), supporting the concept of AC drug inclusion as a novel strategy to mitigate post-DCB restenosis in the presence of EC dysfunction. Case comparisons of the pressure-diameter response (FIG. 6C) of remodeled vessels due to an intermediate degree of hypertension (P^{H}=150 mmHg) show that with respect to the baseline vessel (dashed line), all remodeled vessels exhibit a decrease in distensibility that is more pronounced in maladaptive (Cases 2 -4) as opposed to adaptive scenarios (Case 1). However, even in adaptive remodeling (as defined in our study) and preserved tissue composition, the remodeled vessel exhibits an unfavorable alteration in structural mechanics (increased structural stiffness/reduced compliance) due to the pressure-mediated increase in wall thickness Like the effect on geometrical outcomes of remodeling, the inclusion of an AC drug in the DCB payload shifts the pressure-diameter response after maladaptive remodeling towards the response exhibited after adaptive remodeling (FIG. 6c, Case 4 vs Case 1). To establish further theoretical support for consideration of novel DCBs that co-deliver AP and AC drugs, iterative AC dosing studies were conducted at a fixed, intermediate AP dosing to identify a payload that facilitates preservation of lumen area in maladaptive post-DCB remodeling. We found that when D_{[AP]}=0.5 and D_{[AP]}=0.74, the deformed inner radius after completion of maladaptive post-DCB remains within 3% of the adaptive (baseline) value for the full range of considered P H (FIG. 6D).

### Example 2

### DCB Synthesis, Characterization, and Deployment

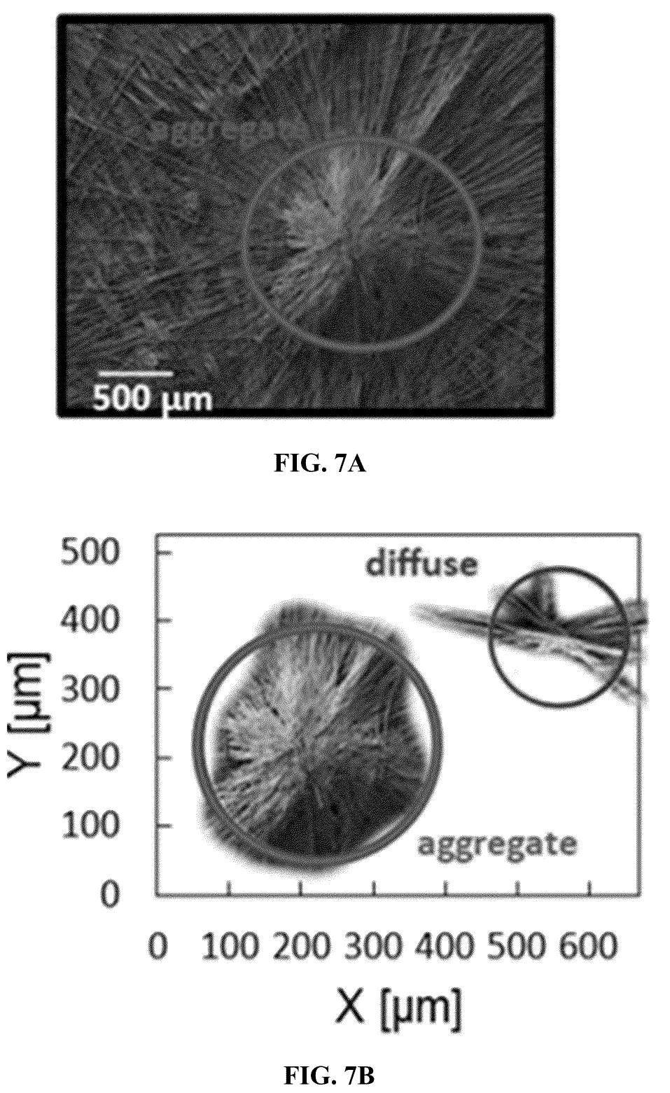

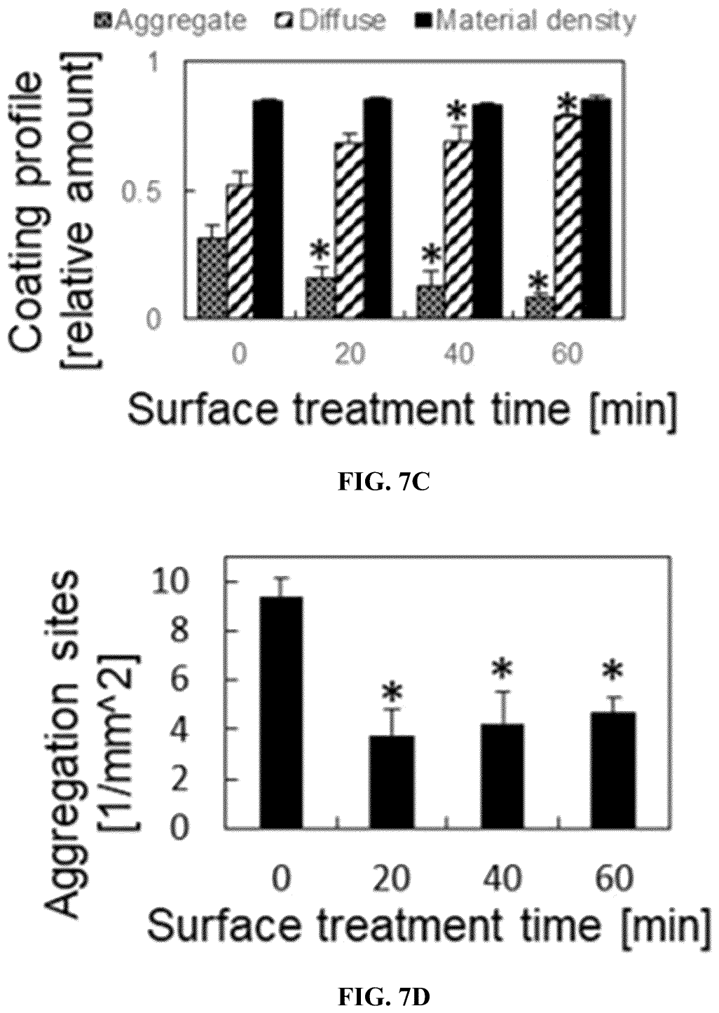

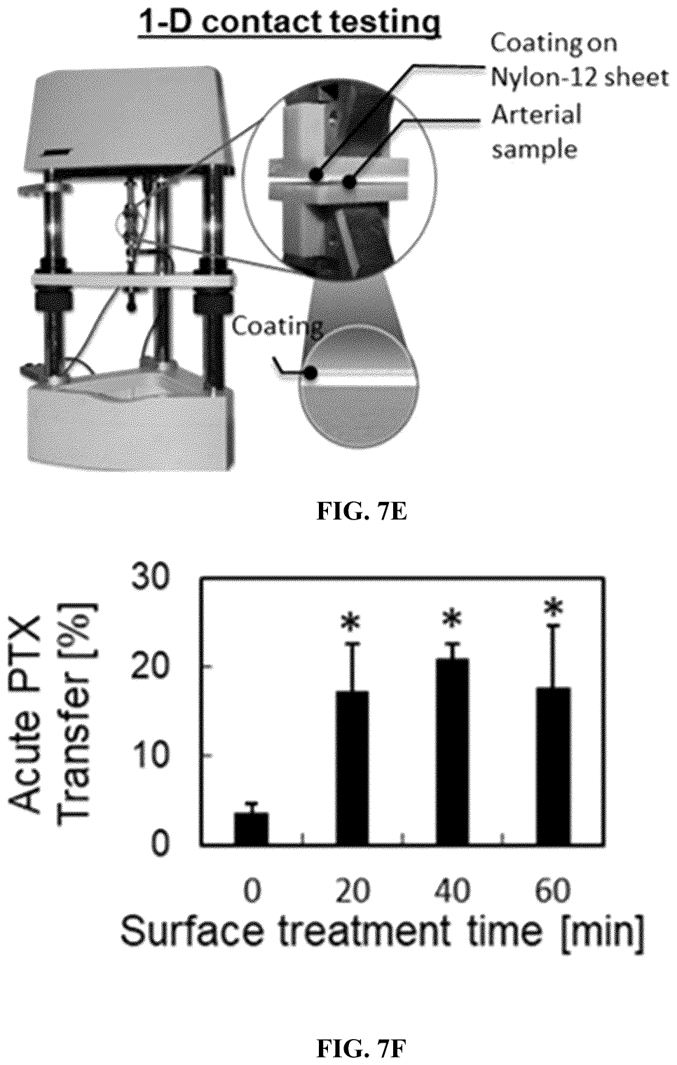

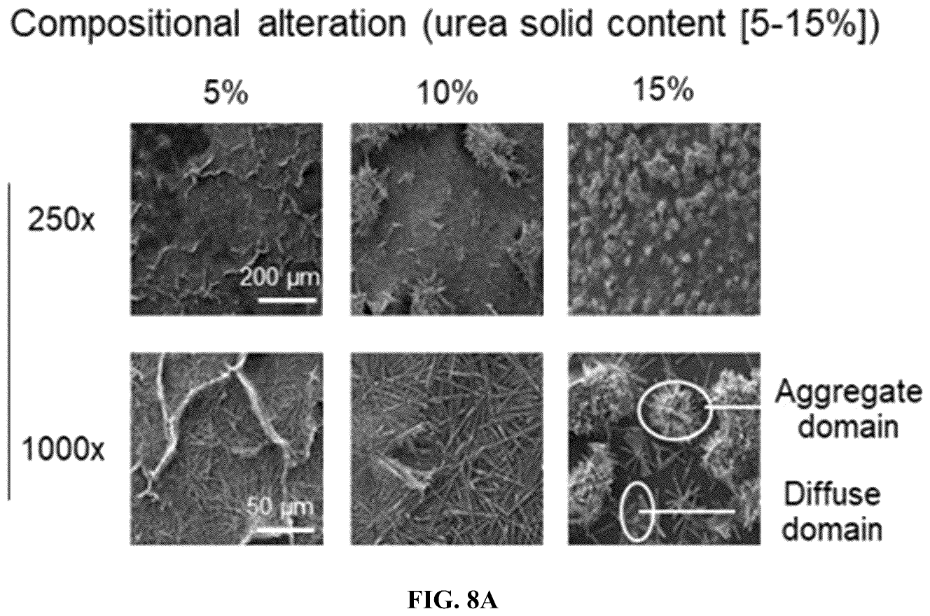

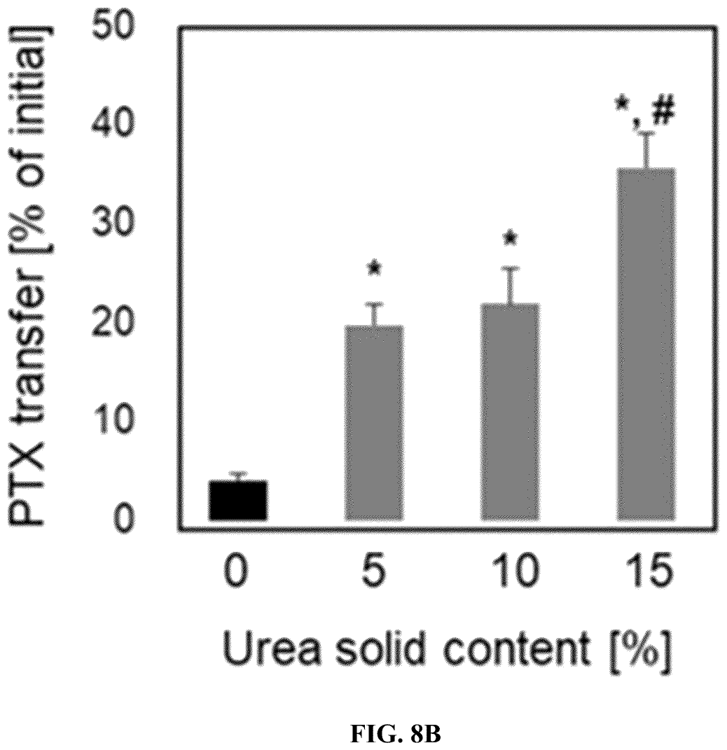

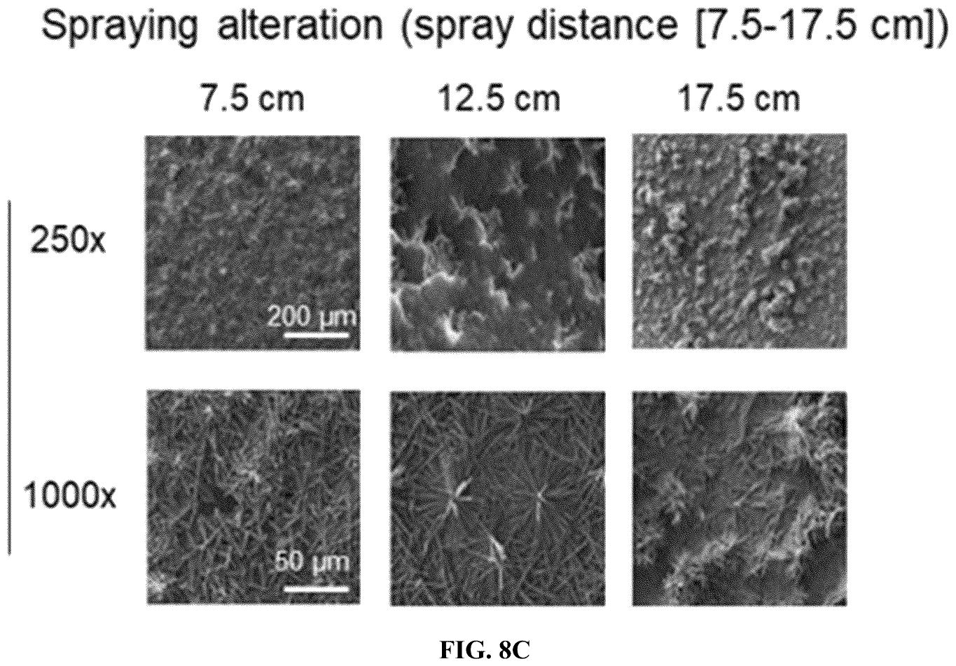

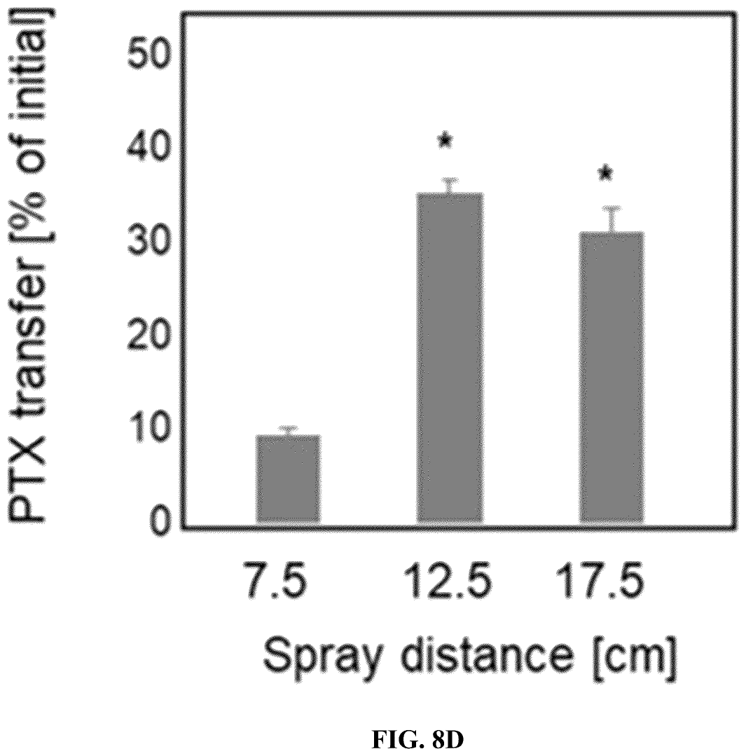

Key biophysical determinants of DCB function and techniques for balloon surface modification prior to coating deposition were identified that promote intra-procedural balloon-coating interfacial failure and coating-tissue adhesion, and quantified PTX transfer efficiency as a function of excipient type. In the context of these studies, semi-automated spray-based methods have been developed for repeatable coating formation and image analysis techniques that provide metrics of coating microstructure which correlate to acute drug transfer (FIGS. 7A-7F). Moreover, a digitized balloon inflation device and associated controls system was constructed that will promote quantifiable and repeatable inflation pressures in proposed ex vivo and in vivo models of DCB deployment and insomuch enhance experiment repeatability. Additionally, urea as the excipient demonstrates that readily tuned coating preparation parameters (i.e., solid content and spraying distance from balloon surface) offer the means to control surface microstructure and PTX transfer to arterial tissue (FIGS. 8A-8B), which may likewise govern drug transfer in co-delivery scenarios.

### Ex-Vivo Studies of Arterial Remodeling

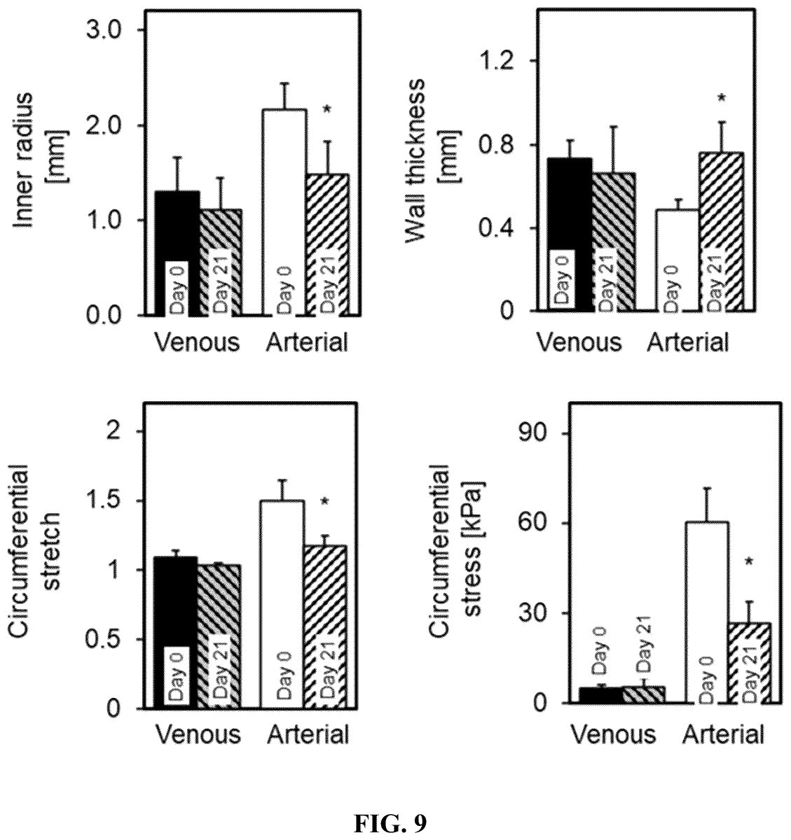

A perfusion bioreactor was constructed and utilized that controls pulsatile pressure, volumetric flow, and axial stretch to deliver precise, mechanomimetic physiological loads to cultured blood vessels (FIGS. 9A-9C). Circulated media consists of DMEM, 10% FBS, supplemented with Penicillin/ Streptomycin, Amphotericin-B, and Gentamycin and is exchanged every 2 days in prolonged (up to 21 days) culture studies. To maintain sterility and pH balance while promoting gas exchange, the system is housed within a sterile incubator maintained at 37 ° C. and 5% CO 2 . More than 70 vessels were successfully cultured in this system and have data generated from 6 hours (n=15), 7 days (n=12), and 21 days (n=10) culture periods, with a variety of porcine vessel types including great saphenous veins (GSVs), radial arteries, internal thoracic arteries, and left anterior descending arteries. Up to 75% of GSVs survive at 21-days of culture and our maximum duration of culture was >4 weeks using renal arteries. During experimentation, vessel viability was assessed using phenylephrine (10⁻⁵ M) and Acetylcholine (10⁻⁶ M) vasoreactants for vSMC cell contractility and EC induced nitric oxide released relaxation, respectively. Endpoint MTT and TUNNEL assays further revealed cultured GSV viability via metabolic activity and apoptosis, respectively. In the study designed to transition GSVs to arterial-like conditions, it was found that tissues achieved a partial restoration of circumferential stretch and circumferential, but not axial, stress through vessel dilation and wall thickening in a primarily outward remodeling process (FIG. 9).

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. An endovascular device, the device comprising:
an outer body comprising a biocompatible polymeric material; and
a core comprising a first layer and a second layer, wherein the first layer comprising an anti-contractile agent and the second layer comprising a second agent.

2. The endovascular device of claim 1, wherein the biocompatible polymeric material comprises polystyrene, poly(lactic acid), polyketal, butadiene styrene, styrene-acrylic-vinyl terpolymer, poly(methyl methacrylate), poly(ethyl methacrylate), poly(alkyl cyanoacrylate), styrene-maleic anhydride copolymer, poly(vinyl acetate), poly(vinyl pyridine), poly(divinylbenzene), poly(butylene terephthalate), acrylonitrile, vinyl chloride-acrylates, poly(ethylene glycol), or a combination thereof.

3. The endovascular device of claim 1, wherein the anti-contractile agent comprises a nitric oxide donor molecule, an angiotensin receptor blocker, or a combination thereof.

4. The endovascular device of claim 1, wherein the anti-contractile agent comprises eprosartan, olmesartan, telmisartan, losartan, valsartan, irbesartan, candesartan, or a combination thereof.

5. The endovascular device of claim 1, wherein the anti-contractile agent comprises valsartan.

6. The endovascular device of claim 1, wherein the second agent is an anti-inflammatory agent, an anti-proliferative agent, or a combination thereof.

7. The endovascular device of claim 6, wherein the anti-inflammatory agent comprises montelukast.

8. The endovascular device of claim 6, wherein the anti-proliferative agent is selected from a group consisting of a cytotoxin or a synthetic molecule or other substances such as actinomycin D, or derivatives and analogs thereof; a taxoid such as taxol, docetaxel, and paclitaxel, paclitaxel derivatives; an olimus drug such as macrolide antibiotics, rapamycin, everolimus, novolimus, myolimus, deforolimus, umirolimus, biolimus, merilimus, temsirolimus structural derivatives and functional analogues of rapamycin (such as 40-O-(3-hydroxy)propyl-rapamycin, 40-O-[2-(2-hydroxy)ethoxy]ethyl-rapamycin, 40-O-tetrazole-rapamycin, or 40-epi-(N1-tetrazolyl)-rapamycin), structural derivatives and functional analogues of everolimus; an mTOR inhibitor; pirfenidone; or a combination thereof.

9. The endovascular device of claim 6, wherein the anti-proliferative agent comprises paclitaxel.

10. The endovascular device of claim 1, wherein the first layer and second layer further comprise urea.

11. The endovascular device of claim 1, wherein the anti-contractile agent and the second agent are present in the endovascular device at a ratio of from about 50:20 to about 20:50.

12. The endovascular device of claim 1, wherein the device is in the form of a cylinder.

13. The endovascular device of claim 1, wherein the device is a stent.

14. The endovascular device of claim 1, wherein the device is a catheter.

15. The endovascular device of claim 1, wherein the device is a balloon.

16. A method of treating peripheral artery disease in a subject in need thereof, the method comprises:
implanting an endovascular device within a peripheral vessel of the subject, wherein the endovascular device comprises an outer body comprising a biocompatible polymeric material and a core comprising a first layer and a second layer, wherein the first layer comprising an anti-contractile agent and the second layer comprising a second agent; and
releasing from the endovascular device a therapeutically effective amount of the anti-contractile agent and the second drug, wherein release of the anti-contractile agent in combination with the second drug preserves the lumen upon maladaptive inward remodeling completion in the subject.

17. The method of claim 16, wherein the anti-contractile agent comprises a nitric oxide donor molecule, an angiotensin receptor blocker, or a combination thereof.

18. The method of claim 16, wherein the anti-contractile agent comprises eprosartan, olmesartan, telmisartan, losartan, valsartan, irbesartan, candesartan, or a combination thereof.

19. The method of claim 16, wherein the anti-contractile agent comprises valsartan.

20. The method of claim 16, wherein the second agent is an anti-inflammatory agent, an anti-proliferative agent, or a combination thereof.

21. The method of claim 20, wherein the anti-inflammatory agent comprises montelukast.

22. The method of claim 20, wherein the anti-proliferative agent is selected from a group consisting of a cytotoxin or a synthetic molecule or other substances such as actinomycin D, or derivatives and analogs thereof; a taxoid such as taxol, docetaxel, and paclitaxel, paclitaxel derivatives; an olimus drug such as macrolide antibiotics, rapamycin, everolimus, novolimus, myolimus, deforolimus, umirolimus, biolimus, merilimus, temsirolimus structural derivatives and functional analogues of rapamycin (such as 40-O-(3-hydroxy)propyl-rapamycin, 40-O-[2-(2-hydroxy)ethoxy]ethyl-rapamycin, 40-O-tetrazole-rapamycin, or 40-epi-(N1-tetrazolyl)-rapamycin), structural derivatives and functional analogues of everolimus; an mTOR inhibitor; pirfenidone; or a combination thereof.

23. The method of claim 20, wherein the anti-proliferative agent comprises paclitaxel.

24. The method of claim 16, wherein the first layer and second layer further comprise urea.

25. The method of claim 16, wherein the anti-contractile agent and the second agent are present in the endovascular device at a ratio of from about 50:20 to about 20:50.

26. The method of claim 16, wherein the device is in the form of a cylinder.

27. The method of claim 16, wherein the device is a stent.

28. The method of claim 16, wherein the device is a catheter.

29. The method of claim 16, wherein the device is a balloon.

30. A modeling framework for predicting the maladaptive remodeling effects of delivery of an anti-proliferative agent to a blood vessel by use of a drug coated balloon (DCB), the modeling framework utilizing the following governing equations:
$$\frac{{P}^{H} {r}_{i ( ma )}}{{h}_{( m a )}} - {σ}_{2 ( b )} = 0$$
$${S}_{( m a )} = \{ \begin{matrix}{S}_{( b )} + ( {S}_{( max )} - {S}_{( b )} ) \frac{{P}^{H} - {P}^{N}}{{P}^{c r} - {P}^{N}} & if {P}^{H} \leq {P}^{cr} \\ {S}_{( max )} & if {P}^{H} > {P}^{cr}\end{matrix}$$
$$2 π ( {r}_{i ( m a )} + \frac{{h}_{( m a )}}{2} ) {h}_{( m a )} - {a}_{( b )} + ( \frac{{S}_{( max )} - {S}_{( m a )}}{{S}_{( max )} - {S}_{( b )}} ) ( 1 - {D}_{[ A P ]} ) ( {a}_{( b )} - {a}_{( a )} ) = 0$$
in which:
p^{H} refers to the hypertensive pressure within the vessel,
p^{N} refers to the normotensive pressure within the vessel,
p^{cr} refers to a critical pressure,
rᵢ refers to the inner radius of the vessel,
s₂ refers to the value for equilibrium in the radial direction of the vessel,
h refers to the vessel wall thickness,
ma (subscript) refers to maladaptive post-DCB remodeling outcomes,
Sₘₐₓ refers to the maximal contractile capacity of the smooth muscle cells of the vessel at a pressure equal to or greater than p″,
S_{b} refers to the basal contractile value of the smooth muscle cells of the vessel at p^{N},
Sₘₐₓ refers to the maladaptive post-DCB remodeling contractile value of the smooth muscle cells of the vessel,
aₐ refers to the cross-sectional wall area of the remodeled artery adaptive remodeling outcome,
a_{b} refers to the basal cross-sectional wall area, and
D_{[AP]} refers to the normalized dosing parameter of the anti-proliferative agent.

31. The method of claim 30, further comprising predicting the effects of an anti-contractile drug co-delivery to the blood vessel by use of the model, the model further including a normalized dosing parameter of the anti-contractile drug (D_{[AP]}), the maladaptive post-DCB remodeling contractile value of the smooth muscle cells of the vessels being modeled as follows:
$${S}_{( m a )} = \{ \begin{matrix}\{ {S}_{( b )} + ( {S}_{( max )} - {S}_{( b )} ) \frac{{P}^{H} - {P}^{N}}{{P}^{c r} - {P}^{N}} \} ( 1 - {D}_{[ A C ]} ) & if {P}^{H} \leq {P}^{cr} \\ {S}_{( max )} ( 1 - {D}_{[ A C ]} ) & if {P}^{H} > {P}^{cr}\end{matrix} .$$
